(19) United States
(12) Patent Application Publication
(10) Pub. No.: US 2009/0209852 A1
(43) Pub. Date: Aug. 20, 2009

(54) Systems and Methods for Treating a Patient Using Guided Radiation Therapy or Surgery

(75) Inventors: Timothy P. Mate, Bellevue, WA (US); Steven C. Dimmer, Bellevue, WA (US); Laurence J. Newell, Mercer Island, WA (US); J. Nelson Wright, Mercer Island, WA (US)

Correspondence Address: PERKINS COIE LLP;PATENT-SEA, P.O. BOX 1247, SEATTLE, WA, 98111-1247, US

(73) Assignee: Calypso Medical Technologies, Inc., Seattle, WA (US)

(21) Appl. No.: 11/817,383

(22) PCT Filed: Mar. 2, 2006
(86) PCT No.: PCT/US06/07508

§ 371 Date: Feb. 3, 2009

Related U.S. Application Data
(60) Provisional application No. 60/658,275, filed on Mar. 2, 2005.

(51) Int. Cl. A61N 5/00; A61B 19/00

(52) U.S. Cl. 600/431; 600/1

(57) ABSTRACT

Systems and methods for locating and tracking a target, i.e., measuring the position and/or rotation of a target during setup and treatment of a patient in guided radiation therapy applications for the head and neck. One embodiment is directed toward a device having a body and markers, such as excitable transponders and/or radiographic fiducials, fixable in or on the body for localizing the body. For example, the body can be a mouthpiece body having a channel configured to receive a patient's teeth such that the mouthpiece is repeatedly and consistently placed in the same relative position in the patient when the patient bites down on the mouthpiece. The transponders can be alternating magnetic transponders and the fiducials can be gold seeds. Other embodiments include a device having a two-piece body, a first piece of the body having excitable transponders and a second piece of the body having radiographic fiducials.

### CROSS-REFERENCE TO RELATED APPLICATION(S)

The present application claims the benefit of U.S. Patent Application No. 60/658,275 filed on Mar. 2, 2005, which is incorporated herein in its entirety.

### TECHNICAL FIELD

This disclosure generally relates to the field of guided radiation therapy, and more particularly, several aspects of the invention are directed toward location markers contained in or on a member configured to be inserted in the cavity of a patient, for example, a mouthpiece for use localizing a tumor or other lesion for head and neck cancer or other medical applications.

### BACKGROUND

Radiation therapy is a common treatment for head and neck cancer. The intention of the therapy is to provide a high dose of radiation to the tumor and a minimal dose to the surrounding normal tissue. Over the past few years, intensity modulated radiation therapy (IMRT) has become the standard of care to perform head and neck irradiation. The dose is delivered over a series of fractions that take several weeks to complete (e.g., up to 40 fractions over 8 weeks), and each treatment may take up to an hour to complete. Because of the high dose gradients delivered by IMRT, it is important for the head to be repositioned accurately within the radiation beam for each of these sessions. The patient's anatomy and position during the course of radiation therapy usually vary to some degree from those used for therapy planning purposes. This is mainly due to patient movement, inaccurate patient positioning, and organ motion.

Setup errors as little as 3 mm in the initial positioning of the patient's head before each treatment (interfractional setup error) can have serious consequences, namely, an insufficient dose coverage of the targeted tumor volume and/or an overdosage of normal tissues. Furthermore, because an IMRT treatment can take up to an hour to complete, patient motion during treatment is also an issue (intrafractional motion). The potential for both interfractional and intrafractional errors to occur increases as treatment progresses because patients become sicker as a result of radiation-induced side effects such as mucositis, fatigue, weight loss, nausea, and thick secretions. These side effects combine to make it increasingly difficult for the patient to remain absolutely still during treatment.

Clinicians employ one of several techniques available for accurately positioning the patient prior to head and neck radiation delivery. The most common technique is to rigidly fix the patient to the treatment table by means of an external fixation device such as a lightweight thermoplastic shell molded over the patient's head and shoulders to form a mask. The thermoplastic mask is then attached to the table and external reference marks on the mask are used to align the patient in the radiation beam by triangulating lasers in the treatment room to the external reference marks. When the external reference marks are in alignment, the assumption is that the patient under the mask also is in the correct position; however, the external reference marks on the thermoplastic mask do not account for movement of the patient's head and shoulders under the mask.

For this setup technique to work, the mask must be expertly molded and fit very snuggly on the patient. Because the mask is molded to the external soft tissues of the patient's cranium and shoulder at the start of treatment and the same mask is used throughout treatment, mask distortion and patient movement under the mask remain a residual problem. Studies have shown that while thermoplastic masks reduce interfractional setup error versus the absence of thermoplastic masks, setup errors of 3 mm or more can still occur daily in 40% of the patients.

Another limitation of thermoplastic masks is the inability to determine whether the patient moves under the mask during treatment because the radiation therapist is outside the treatment room. Given that a typical head and neck IMRT treatment can take up to an hour, patient drift under the mask is a problem. Potential movement becomes even more problematic as treatment progresses due to patient weight loss and loosening of the mask.

To further improve upon thermoplastic mask fixation, additional localization devices have been designed and approved for clinical use in conjunction with the mask. Most notable is the category of custom dental mold devices with an extra-oral extension outfitted with infrared, ultrasound, or radiographic detectors that can be located by respective detection systems installed in the treatment room. The custom oral dental mold is positioned on the maxillary teeth and further fixes to the thermoplastic mask to the patient. The extra-oral portion of the dental mold is located by the detection system. The custom molded dental mold fixes to the skull by fixing to the teeth, and hence the skull position is registered to the treatment room by registering the extra-oral portion of the dental mold. At the beginning of each radiation session, the detected skull position is compared to the reference baseline position. Any discrepancy between the two may be reconciled by making an adjustment in the treatment table position to which the patient is fixed. Although localization systems based on a custom mouthpiece used in conjunction with a thermoplastic mask can further reduce interfractional setup error, they do not address intrafractional motion.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been solely selected for ease of recognition in the drawings.

FIG. 1 is an isometric view schematically illustrating a mouthpiece body having a channel for receiving a patient's teeth and excitable markers embedded in the mouthpiece in accordance with an embodiment of the invention.

FIG. 2 is a front elevation view schematically illustrating the mouthpiece body of FIG. 1 in accordance with an embodiment of the invention.

FIG. 3 is a side elevation view schematically illustrating the mouthpiece body of FIG. 1 in accordance with an embodiment of the invention.

FIG. 4 is an isometric view schematically illustrating a mouthpiece body having a channel for receiving a patient's teeth and radiographic fiducials embedded in the mouthpiece in accordance with an embodiment of the invention.

FIG. 5 is a top view schematically illustrating a mouthpiece having a channel for receiving a patient's teeth; the mouthpiece body includes excitable markers and radiographic fiducials embedded in the mouthpiece in accordance with an embodiment of the invention.

FIG. 6 is a front elevation view schematically illustrating the mouthpiece of FIG. 4; the mouthpiece body is a two-piece device in accordance with an embodiment of the invention.

FIG. 7 is an exploded side elevation view schematically illustrating the two-piece mouthpiece body of FIG. 5 in accordance with an embodiment of the invention.

FIG. 8 is a side elevation view schematically illustrating a tracking system for use in localizing and monitoring a target in accordance with an embodiment of the present invention; excitable markers are shown embedded in a mouthpiece and placed in a patient's oral cavity and adjacent to a target in the patient in accordance with an embodiment of the invention.

FIG. 9 is a schematic elevation view of the patient on a movable support table and of markers within a mouthpiece body and placed in a patient's oral cavity in accordance with an embodiment of the invention.

FIG. 10 is a side view schematically illustrating a localization system and a plurality of markers within a mouthpiece body and placed in a patient's oral cavity in accordance with an embodiment of the invention.

FIG. 11 is a flow diagram of an integrated radiation therapy process that uses real-time target tracking for radiation therapy in accordance with an embodiment of the invention.

FIG. 12A is a representation of a CT image illustrating an aspect of a system and method for real-time tracking of targets in radiation therapy and other medical applications.

FIG. 12B is a diagram schematically illustrating a reference frame of a CT scanner.

FIG. 13 is a screenshot of a user interface for displaying an objective output in accordance with an embodiment of the invention.

FIG. 14 is an isometric view of a radiation session in accordance with an embodiment of the invention.

FIG. 15A is an isometric view of a marker for use with a localization system in accordance with an embodiment of the invention.

FIG. 15B is a cross-sectional view of the marker of FIG. 14B taken along line 15B-15B.

FIG. 15C is an illustration of a radiographic image of the marker of FIGS. 14A-14B.

FIG. 16A is an isometric view of a marker for use with a localization system in accordance with another embodiment of the invention.

FIG. 16B is a cross-sectional view of the marker of FIG. 15A taken along line 16B-16B.

FIG. 17A is an isometric view of a marker for use with a localization system in accordance with another embodiment of the invention.

FIG. 17B is a cross-sectional view of the marker of FIG. 16A taken along line 17B-17B.

FIG. 18 is an isometric view of a marker for use with a localization system in accordance with another embodiment of the invention.

FIG. 19 is an isometric view of a marker for use with a localization system in accordance with yet another embodiment of the invention.

FIG. 20 is a schematic block diagram of a localization system for use in tracking a target in accordance with an embodiment of the invention.

FIG. 21 is a schematic view of an array of coplanar source coils carrying electrical signals in a first combination of phases to generate a first excitation field.

FIG. 22 is a schematic view of an array of coplanar source coils carrying electrical signals in a second combination of phases to generate a second excitation field.

FIG. 23 is a schematic view of an array of coplanar source coils carrying electrical signals in a third combination of phases to generate a third excitation field.

FIG. 24 is a schematic view of an array of coplanar source coils illustrating a magnetic excitation field for energizing markers in a first spatial orientation.

FIG. 25 is a schematic view of an array of coplanar source coils illustrating a magnetic excitation field for energizing markers in a second spatial orientation.

FIG. 26A is an exploded isometric view showing individual components of a sensor assembly for use with a localization system in accordance with an embodiment of the invention.

FIG. 26B is a top plan view of a sensing unit for use in the sensor assembly of FIG. 26A.

FIG. 27 is a schematic diagram of a preamplifier for use with the sensor assembly of FIG. 26A.

### DETAILED DESCRIPTION

In the following description, certain specific details are set forth in order to provide a thorough understanding of various embodiments of the invention. However, one skilled in the relevant art will recognize that the invention may be practiced without one or more of these specific details, or with other methods, components, materials, etc. In other instances, well-known structures associated with target locating and tracking systems have not been shown or described in detail to avoid unnecessarily obscuring descriptions of the embodiments of the invention.

Unless the context requires otherwise, throughout the specification and claims which follow, the word “comprise” and variations thereof, such as “comprises” and “comprising,” are to be construed in an open, inclusive sense that is as “including, but not limited to.”

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

The headings provided herein are for convenience only and do not interpret the scope or meaning of the claimed invention.

### A. Overview

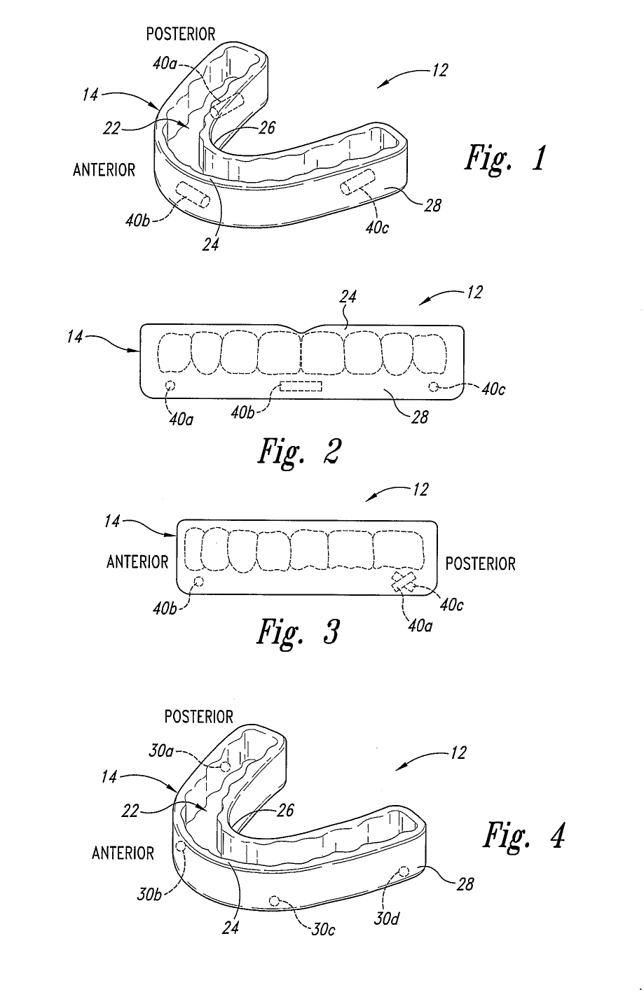

Targeting of cancer therapy in the head and neck area of the body requires increased accuracy due to critical structures that may be located adjacent to cancerous lesion treatment targets. FIGS. 1-27 illustrate a system and several components for locating, tracking and monitoring a target within a patient in real time in accordance with embodiments of the present invention. The system and components guide and control the radiation therapy to more effectively treat the target. Several embodiments of the systems described below with reference to FIGS. 1-27 can be used to treat targets in the head, neck, cervical, prostate and other parts of the body in accordance with aspects of the present invention. Additionally, the markers and localization systems shown in FIGS. 1-27 may also be used in surgical applications or other medical applications. Like reference numbers refer to like components and features throughout the various figures.

The present disclosure describes devices, systems, and methods for locating and tracking a target, i.e., measuring the position and/or rotation of a target during setup and treatment of a patient in medical applications, for example, in head and neck radiation therapy applications. A patient positioning system for head and neck radiation therapy applications requires greater 3D localization accuracy than many other cancer sites due to the close proximity of radiation-sensitive organs to the radiation treatment volume.

Several aspects of the invention are related to a device having a body and markers, such as excitable transponders, fixable in or on the body for localizing the body. The body can be configured to be releasably secured at the same location of the patient repeatedly. For example, the body can be a mouthpiece molded to fit the oral cavity of the patient such that the mouthpiece is consistently placed in the same relative position in the patient when the patient bites down on the mouthpiece. According to other aspects, the body can be a conformal member, a reciprocal member, a probe, a tube, an intubation device or any other device insertable into a body cavity for use in radiation therapy locating and/or tracking the position and/or rotation of a target during diagnosis, planning, setup and treatment of a patient in medical applications. The transponders can be alternating magnetic transponders having a core, a coil around the core, and an optional capacitor coupled to the coil. In several applications, one or more transponders are carried in or on the body.

One aspect is directed toward a device having a body and markers, such as excitable transponders and/or radiographic fiducials, fixable in or on the body for localizing the body. For example, the body can be a mouthpiece body having a channel configured to receive a patient's teeth such that the mouthpiece is repeatedly and consistently placed in the same relative position in the patient when the patient bites down on the mouthpiece. The transponders, for example, can be alternating magnetic transponders, and the fiducials can, for example, be gold seeds or other radiographic materials. One skilled in the art will recognize that the transponders and/or the fiducials need not be limited to those described here. Further aspects include a plurality of transponders at known location and orientation to one another.

One aspect is directed to a device having a two-piece body, a first piece of the body having excitable transponders and a second piece of the body having radiographic fiducials. The first piece and the second piece of the body can releasably couple to form a mouthpiece, and the transponders and fiducials can be fixable in or on the body. Further aspects include that either the first piece or the second piece of the body can be a mouthpiece and the other of the first or second piece may be an insert releasably coupled to the mouthpiece. The mouthpiece can be configured to be releasably retained in an oral cavity of a patient. According to aspects of the invention, the body can be configured to be releasably retained in any cavity of a patient. Further aspects include a plurality of transponders in known location and orientation to one another and/or in known location and orientation to the radiographic fiducials.

In operation, the body is releasably retained in a cavity of the patient and the relative positions between the transponders and/or fiducials and the lesion are determined using imaging techniques. During a setup process and/or the application of therapy radiation, the body is reattached to the patient. The transponders are localized using an alternating magnetic field and the fiducials are localized using CT or MR imaging. Based upon the measured positions of the transponders and the predetermined relative positions between the transponders and the lesion when the body is attached to the patient, the location of the lesion relative to a reference frame and/or the radiation beam is determined in real time during the setup procedure and/or the application of therapy radiation.

Several embodiments of the invention are directed toward methods for tracking a target, i.e., measuring the position and/or the rotation of a target in substantially real time, in a patient in medical applications. One embodiment of such a method comprises collecting position data of a marker that is substantially fixed relative to the target. This embodiment further includes determining the location of the marker in an external reference frame (i.e., a reference frame outside the patient) and providing an objective output in the external reference frame that is responsive to the location of the marker. The objective output is repeatedly provided at a frequency/periodicity that adequately tracks the location of the target in real time within a clinically acceptable tracking error range. As such, the method for tracking the target enables accurate tracking of the target during diagnostic, planning, setup, treatment, or other types of medical procedures. In many specific applications, the objective output is provided within a suitably short latency after collecting the position data and at a sufficiently high frequency to use the data for such medical procedures.

Another specific embodiment is a method for treating a target in a patient with an ionizing radiation beam that includes collecting position information of a marker fixable in or on a body, the body positioned within a patient at a site relative to the target at a time tₙ, and providing an objective output indicative of the location of the target based on the position information collected at time tₙ. The objective output is provided to a memory device, user interface, and/or radiation delivery machine within 2 seconds or less of the time tₙ when the position information was collected. This embodiment of the method can further include providing the objective output at a periodicity of 2 seconds or less during at least a portion of a treatment procedure. For example, the method can further include generating a beam of ionizing radiation and directing the beam to a machine isocenter, and continuously repeating the collecting procedure and the providing procedure every 10-200 ms while irradiating the patient with the ionizing radiation beam.

Another embodiment of a method for tracking a target in a patient includes obtaining position information of a marker fixable in or on a body, the body situated within the patient at a site relative to the target, and determining a location of the marker in an external reference frame based on the position information. This embodiment further includes providing an objective output indicative of the location of the target to a user interface at (a) a sufficiently high frequency so that pauses in representations of the target location at the user interface are not readily discernable by a human, and (b) a sufficiently low latency to be at least substantially contemporaneous with obtaining the position information of the marker.

Another embodiment of the invention is directed toward a method of treating a target of a patient with an ionizing radiation beam by generating a beam of ionizing radiation and directing the beam relative to the target. This method further includes collecting position information of a marker fixable in or on a body, the body placed within the patient at a site relative to the target while directing the beam toward the beam isocenter. Additionally, this method includes providing an objective output indicative of a location of the target relative to the beam isocenter based on the collected position information. This method can further include correlating the objective output with a parameter of the beam, and controlling the beam based upon the objective output. For example, the beam can be gated to only irradiate the patient when the target is within a desired irradiation zone. Additionally, the patient can be moved automatically and/or the beam can be shaped automatically according to the objective output to provide dynamic control in real time that maintains the target at a desired position relative to the beam isocenter while irradiating the patient.

Various embodiments of the invention are described in this section to provide specific details for a thorough understanding and enabling description of these embodiments. A person skilled in the art, however, will understand that the invention may be practiced without several of these details, or that additional details can be added to the invention. Where the context permits, singular or plural terms may also include the plural or singular term, respectively. Moreover, unless the word “or” is expressly limited to mean only a single item exclusive from the other items in reference to a list of at least two items, then the use of “or” in such a list is to be interpreted as including (a) any single item in the list, (b) all the items in the list, or (c) any combination of items in the list. Additionally, the term “comprising” is used throughout to mean including at least the recited feature(s) such that any greater number of the same feature and/or types of other features or components are not precluded.

### B. Instruments for Head and Neck Procedures

FIG. 1 is an isometric view of a localization device 12 including a body 14 and a plurality of markers 40a-c in accordance with an embodiment of the invention. The body 14 may be a mouthpiece molded to fit the oral structure, for example, the maxillary teeth, or mandibular teeth of a patient. The markers 40a-c may be excitable transponders. Suitable markers include those disclosed in U.S. patent application Ser. No. 09/954,700, filed Sep. 14, 2001; U.S. Pat. No. 6,812,842, issued Nov. 2, 2004; U.S. patent application Ser. No. 09/877,498, filed on 8 Jun. 2001; U.S. patent application Ser. No. 11/166,801, filed Jun. 24, 2005; U.S. Pat. No. 6,838,990, issued Jan. 4, 2005; and U.S. Pat. No. 6,822,570, issued Nov. 23, 2004, hereby incorporated by reference in their entirety.

The body 14 can have other configurations in other embodiments; for example, the body 14 may be a conventional mouth guard, bite block, bite splint, or the like. Suitable mouth guards include those disclosed in U.S. Pat. Nos. 4,791,941, 3,211,143, 2,630,117, 3,224,441, 3,124,129, 3,096,761, 3,112,744, hereby incorporated by reference in their entirety. The body 14 is generally configured so that it can be releasably secured to the patient in the same position with a high degree of repeatability. The body 14 can accordingly be held in the desired position on the patient for a treatment fraction, removed from the patient between treatment fractions, and then reinstalled at the same position for a subsequent treatment fraction over a large number of treatment fractions.

According to further embodiments, the body may be a conformal member configured to be releasably retained in a cavity of a patient. For example, the body-cavity probe having a confirmed member disclosed in U.S. Pat. No. 6,625,495, hereby incorporated in its entirety by reference, may be used in accordance with this invention. Alternatively, a conformal member may be constructed out of thermoplastic materials to provide a conformal member having defined reciprocal characteristics of the patient's cavity. In yet another embodiment, suitable conformal members may be a catheter, tube, probe or intubation device. One example of an intubation device that may be used in combination with the localization system is disclosed in U.S. Pat. No. 5,897,521, hereby incorporated in its entirety by reference.

The body 14 may be formed of a thermoplastic material or like materials. A body constructed of thermoplastic materials can be heated prior to initial use to mold the body to the patient's specific bite, maxillary (upper) teeth or mandibular (lower) teeth and thus provide a custom mouthpiece body. A custom mouthpiece body provides a greater degree of accuracy for the localization system. Alternatively, the body 14 may be formed of semi-rigid rubber, plastic, ceramic, polymeric, rigid plastic, composites, or like materials. Alternatively, when the body 14 is molded to the maxillary teeth, it may further include a partial or full impression of the mandibular teeth on an underside of a base plate 28 to further prevent movement of the patient's jaw. Correspondingly, when the body 14 is molded to the mandibular teeth, it may further include a partial or full impression of the maxillary teeth on an underside of the base plate 28 to further prevent movement of the patient's jaw. A suitable mouthpiece having a dual impression includes that disclosed in U.S. Pat. No. 3,250,272, hereby incorporated by reference in its entirety.

FIG. 4 shows an isometric view of a localization device 12 including a body 14 and a plurality of fiducials 30a-d. The fiducials 30a-d may be radiographic or radiopaque fiducials as is known in the art. The radiographic fiducials' 30 design may be any one of the following examples: small metal spheres, small diameter metal wire or metal wire formed into a crosshair and the like. The radiographic fiducial can be made from gold, tungsten, platinum and/or other high density metals. According to certain embodiments, a set of radiographic fiducials 30 are provided with geometries that are optimized for localization with imaging devices (e.g., CT or MRI).

The body 14 shown in FIGS. 1-4 is generally unshaped and includes a u-shaped channel 22 for seating teeth of the patient therein. The channel 22 may have a first and a second wall 24, 26 extending upwardly from the base plate 28 to form the channel 22. The transponders 40a-c are fixedly positioned in the base plate 28 in the exemplary embodiment; however, it is understood that the transponders may be fixedly positioned in or on the first wall 24, in or on the second wall 26, or in or on a combination of the first wall 24, the second wall 26, or the base plate 28.

The transponders 40a-c are preferably small markers such as alternating magnetic transponders. The transponders 40a-c can each have a unique frequency relative to each other to allow for time and frequency multiplexing. The transponders 40a-c can accordingly include a core, a coil wound around the core, and a capacitor electrically coupled to the coil. A localization device 12 can include one or more transponders 40, and as such is not limited to having three transponders 40a-c as illustrated. The transponders are localized using a source, sensor array, receiver, and localization algorithm as described further herein.

In operation, the three transponders may be used to localize a treatment target isocenter relative to a linear accelerator radiation therapy treatment isocenter. The treatment target localization may include both translational offset (X, Y, and Z directions) and a rotational offset (pitch, yaw, and roll) relative to a linear accelerator coordinate reference frame.

FIGS. 1-3 show three transponders 40a-c embedded in a base plate 28 of the mouthpiece body 14. The transponders 40a-c are used to localize the mouthpiece body 14 and resulting patient target treatment isocenter relative to the linear accelerator machine isocenter. As a process step during radiation therapy treatment planning, a patient undergoes a CT scan whereby the X, Y, and Z positions of the radiographic centers for all three transponders 40a-c as well as the X, Y, and Z position for the treatment target isocenter are identified. To localize a patient treatment target isocenter relative to the linear accelerator treatment target isocenter both prior to and during radiation therapy delivery, the three transponder positions that are fixable in or on a mouthpiece body 14 are localized electromagnetically and then used to calculate the position of the treatment target isocenter position and rotational offsets.

In accordance with this embodiment of the invention, accuracy of a transponder centroid localization in computed tomography (CT) may limit the accuracy that the transponders 40a-c in or on the mouthpiece body 14 are localized to and thus may limit the accuracy of the resulting translational and rotational treatment isocenter offset accuracy. Further, rotational offset localization accuracy may be limited due to the spacing geometry between the transponders 40a-c.

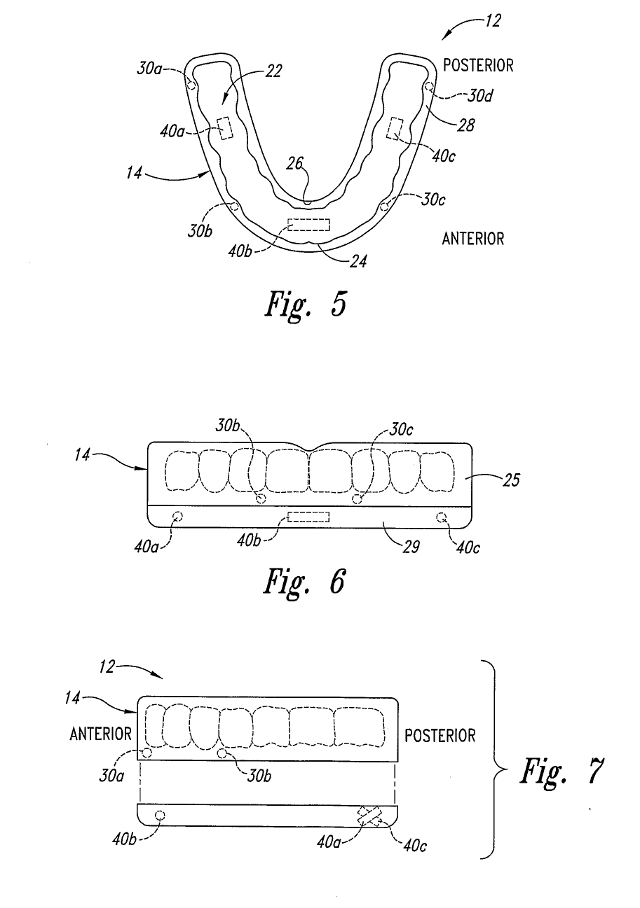

According to further embodiments, the accuracy of transponder centroid localization in CT may therefore be improved by the addition of radiographic fiducials 30a-d in the mouthpiece body 14. FIG. 5 shows a mouthpiece having a channel 22 for receiving a patient's teeth (not shown); the mouthpiece body 14 includes excitable markers 40a-c and radiographic fiducials 30a-d. In certain embodiments, the mouthpiece body 14 includes transponders 40a-c and/or radiographic fiducials 30a-d that are positioned at known locations relative to each other. The design of the radiographic fiducials 30 design may be any one of the following examples: small metal spheres, small diameter metal wire, or metal wire formed into a crosshair and the like. An increased number of radiographic fiducials 30 will increase the localization accuracy since the localization accuracy of each fiducial 30 is independent of the other fiducials 30 and the accuracy of the body 14 localization is essentially dependent on the average accuracy of the fiducial localization. According to certain embodiments, a set of radiographic fiducials 30 are provided with geometries that are optimized for localization with imaging devices (e.g., CT or MRI).

According to further embodiments and as shown in FIG. 6, the radiographic fiducials 30a-d may be fixable in or on a first piece 25 of the body 14 and the magnetic transponders 40 may be fixable in or on a second detachable piece 29 of the body 14. According to this embodiment, imaging and treatment planning with magnetic resonance imaging (MRI) is enabled using fiducials that are compatible with MRI (i.e., they do not create image artifacts). The two pieces (one with radiographic fiducials and one with transponders) could be joined together after treatment planning and used for localization during radiation therapy. The first and second pieces 25, 29 of the body 14 may be coupled together by snaps, clasps, tongue and groove, or other mechanical connections as is known in the art.

In operation, the first piece 25 containing radiographic fiducials 30 may be inserted in a patient's oral cavity during treatment planning. During treatment planning, a CT or MR image is typically used to locate the fiducials 30 and plan the treatment. The second piece 29 of the body 14 containing the transponders 40 can then be coupled to the first piece 25 prior to treatment. During treatment, the transponders are excited and localized as described herein. Placing the second piece 29 after the first piece 25 prevents image artifacts from being created during MRI.

In yet another embodiment of the invention, adequate distance between the transponders 40 and radiographic fiducials 30 is maintained so that MRI artifacts created by the presence of transponders 40 do not infer with the localization of the radiographic fiducials 30 by MRI. Accordingly, the mouthpiece body may be one-piece, two-piece, or multi-piece construction. In yet another embodiment of the invention, the rotational offset localization accuracy may be improved by designing a mouthpiece body that incorporates transponders that are positioned at known orientations relative to each other; therefore, the mouthpiece body may includes transponders 40 and radiographic fiducials 30 that are positioned at known locations relative to each other.

In operation, the geometry of the head and neck radiation therapy treatment places further demands on the patient positioning system. Localization of a target, namely, the treatment isocenter or the patient treatment volume, near the centroid of the three transponders in the body does not require knowledge of the rotational orientation of the body. However, in head and neck radiation therapy or other treatment applications, portions of the patient treatment volume can be far removed from the body. Accurate 3D localization of a target removed from the centroid of the three transponders 40 requires accurate knowledge of the rotational orientation of the body 14 in addition to the translational orientation of the body 14. A patient positioning system for head and neck treatment applications, therefore, may further benefit from accurate 6D tracking of the body 14 including the three dimensions of rotation, namely, pitch, yaw, and roll, in addition to the three dimensions of translation, namely X, Y, and Z.

In certain embodiments, rotational orientation may be determined by comparing the 3D locations of the transponders 40 as measured by the localization system to the 3D locations of the transponders 40 as determined in treatment planning, usually with a CT imaging system. Rotational orientation is determined at patient setup because at patient setup the linear accelerator gantry is positioned under the patient and therefore does not interfere with the magnetic localization of the transponders 40. According to one embodiment, during treatment, the localization system enters a translate-only or centroid-tracking mode of localization (3D tracking). As the linear accelerator gantry swings overhead close to an array of the localization system, localization accuracy of one or more transponders 40 may become degraded. In some cases, a transponder's location may be rendered unmeasurable or of unacceptable accuracy by narrow-band interfering sources in the gantry. As further described below, the localization system can dynamically assign weights to the plurality of transponders 40 based on the quality of the transponder signal, and thereby disregard unreliable transponder signals. In addition, the localization system can accurately track the centroid of the three transponders 40 (assuming the same fixed rotational orientation determined at patient setup) with as little as one quality transponder signal.

In embodiments directed to 6D tracking, for example in head and neck treatment, it may be desirable to determine rotational orientation throughout the treatment, including when the gantry is in proximity to the array. If the 6D orientation of the body is determined solely from the 3D locations of each of the transponders 40 (point-based registration) and if one transponder location has been rendered unmeasurable by external interference, the 6D orientation of the body may not be determinable. Alternatively, if one of the transponder locations has been rendered inaccurate by external interference, the 6D orientation of the body may be inaccurate.

Therefore, in addition to determining the location of each transponder 40, the localization system can accurately and precisely determine the orientation of each transponder 40. The transponder signal, however, is invariant under rotations about the transponder axis. Thus, the localization system cannot provide any information about transponder rotation about an axis parallel to the transponder's axis, but it can provide accurate information about the other two degrees of freedom for rotation. If diversity in transponder orientations can be built into the body, the body's rotational orientation can be determined by measuring the transponders' orientations. Further, as shown in FIGS. 5-7, if each of the three transponders 40 is placed orthogonal to one another, the body's rotational orientation can be determined with any two measurable transponders. Accordingly, rotational orientation may be determined at each 100 msec measurement throughout the treatment, yielding a more robust localization system.

One expected advantage of the localization device 12 is that estimates of the rotational orientation of the body can be improved in both precision and accuracy, thus, (1) providing improved speed and accuracy of the repositioning data by providing objective data and eliminating the need for manual identification of anatomical landmarks and (2) providing full characterization of the translation and rotation of the target. Additionally, the robustness of the localization system in the presence of “worst case” electromagnetic environments is greatly improved for 6D tracking applications. For example, a localization system having transponders in known location and orientation relative to one another would retain the ability to provide accurate 6D tracking information even in the event that the orientation and/or location of one transponder is rendered unmeasurable or inaccurate. Thus, the localization system would estimate the body's orientation by looking at the positions of the transponders and the orientation of each transponder and weighting this data appropriately.

Another expected advantage of the localization device 12 is the elimination of an external fixation device, for example, the thermoplastic mask. The transponders in the body can be localized and tracked during treatment, thus allowing a simple support device under or around the patient's head and shoulders to provide sufficient positioning support. Alternatively, the localization device 12 can be used in conjunction with the thermoplastic mask to provide greater localization accuracy and efficiency during setup and/or tracking during radiation treatment.

Another expected advantage of the localization system is insertion of the conformal member having markers contained in or on the conformal member into a body cavity (e.g. ear, nasal, oral, vaginal, rectal, urethral) to localize a target in a patient during diagnosis, setup, planning and radiation treatment of the patient. For example, a conformal member can be inserted into a vaginal cavity during diagnosis, setup, planning and/or radiation treatment of a cervical lesion. Alternatively, a conformal member can be inserted into a rectal cavity during diagnosis, setup, planning and/or radiation treatment of a colon lesion. Alternatively, a conformal member can be inserted into an ear canal, nasal, or oral cavity during diagnosis, setup, planning and/or radiation treatment of a head and neck lesion.

Another expected advantage is the ability to register different imaging modalities to one common platform, for example, Positron Emission Tomography (PET), CT, and MRI scans can be registered to one platform according to aspects of the invention. Registering multiple modalities to one common platform can result in many efficiency and accuracy advantages in diagnosis, planning an treatment phases of the patient's therapy.

The marker orientation is affected by the orientation diversity of the markers and the mechanical tolerances of the material the body is constructed from. With regard to orientation diversity, it is advantageous for the three transponders 40a-c to be placed orthogonal to one another such that the three degrees of freedom of body rotation can be determined even if only two transponders are measurable, as shown best in FIGS. 3 and 6. Two degrees of freedom of rotation is obtained from each transponder. If the two measurable transponders are in the same orientation, only two degrees of freedom is determined from transponder orientation. FIGS. 5-7 show one illustrative layout of attaining this relative orientation. As will be understood by those skilled in the art, the transponders can also be placed in alternative known orientations.

With regard to mechanical tolerances of body construction, the body can be “hard-coded” into the localization system software if the body is constructed with tight position (<0.25 mm) and orientation (<0.5 degree) tolerances. Alternately, the relative positions and orientations of the transponders, and/or radiographic fiducials, could be determined in the treatment planning process.

The relative positions of the markers, radiographic and/or magnetic, and critical features of the patient's anatomy are typically determined in the treatment planning process. This process typically consists of locating the fiducials and anatomy features on a CT scan. However, while the position of the transponders or the positions of radiographic fiducials can be determined fairly accurately (˜0.5 mm) in a CT image, the rotational orientation of the transponders cannot be accurately determined from a CT image.

According to aspects of the invention, the orientation of the transponders can be accurately known by construction. If the body is not manufactured to this degree of accuracy, the orientation of the transponders could be registered to the transponder locations using the localization system. This could be done at patient setup in the absence of interferers, or metal from the gantry, or during a separate “treatment planning” session with the localization system.

C. Radiation Therapy Systems with Real-Time Tracking Systems

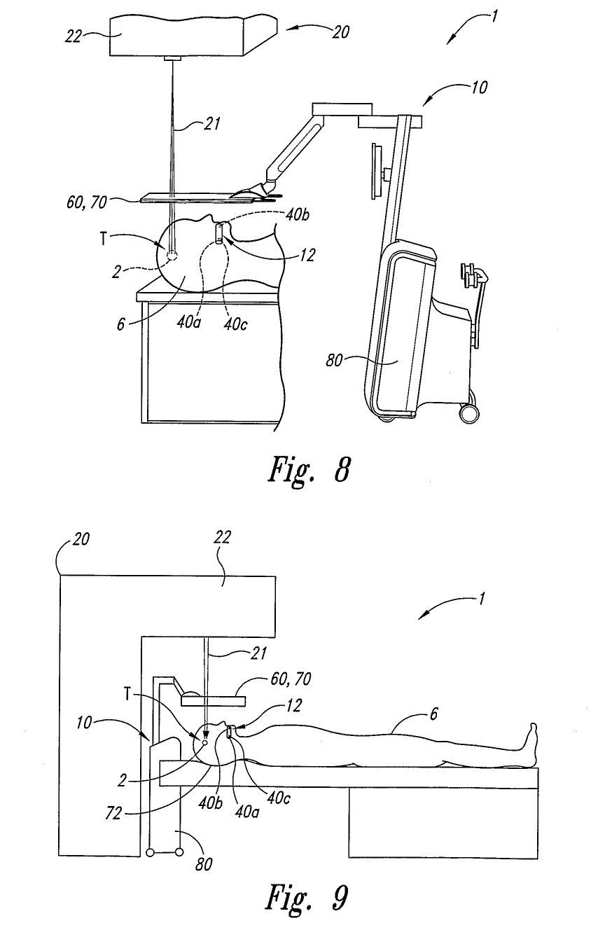

FIGS. 8 and 9 illustrate various aspects of a radiation therapy system 1 for applying guided radiation therapy to a target 2 (e.g., a tumor) within a head, neck or other part of a patient 6. The radiation therapy system 1 has a localization system 10 and a radiation delivery device 20. The localization system 10 is a tracking unit that locates and tracks the actual position of the target 2 in real time during treatment planning, patient setup, and/or while applying ionizing radiation to the target from the radiation delivery device. Moreover, the localization system 10 continuously tracks the target and provides objective data (e.g., three-dimensional coordinates in an absolute reference frame) to a memory device, user interface, linear accelerator, and/or other device. The system 1 is described below in the context of guided radiation therapy for treating a tumor or other target in the head and neck of the patient, but the system can be used for tracking and monitoring other targets within the patient for other therapeutic and/or diagnostic purposes.

The radiation delivery source of the illustrated embodiment is an ionizing radiation device 20 (i.e., a linear accelerator). Suitable linear accelerators are manufactured by Varian Medical Systems, Inc. of Palo Alto, Calif.; Siemens Medical Systems, Inc. of Iselin, N.J.; Elekta Instruments, Inc. of Iselin, N.J.; or Mitsubishi Denki Kabushik Kaisha of Japan. Such linear accelerators can deliver conventional single or multi-field radiation therapy, 3D conformal radiation therapy (3D CRT), IMRT, stereotactic radiotherapy, and tomo therapy. The radiation delivery device 20 can deliver a gated, contoured, or shaped beam 21 of ionizing radiation from a movable gantry 22 to an area or volume at a known location in an external, absolute reference frame relative to the radiation delivery device 20. The point or volume to which the ionizing radiation beam 21 is directed is referred to as the machine isocenter.

The tracking system includes the localization system 10 and one or more markers 40. The localization system 10 determines the actual location of the markers 40 in a three-dimensional reference frame, and the markers 40 are typically within the patient 6. In the embodiment illustrated in FIGS. 8 and 9, more specifically, three markers identified individually as markers 40a-c are in or on a body 14 positioned in an oral cavity of the patient 6 at locations in or near the target 2. In other applications, a single marker, two markers, or more than three markers can be used depending upon the particular application. The markers 40 are desirably placed relative to the target 2 such that the markers 40 are at least substantially fixed relative to the target 2 (e.g., the markers move at least in direct proportion to the movement of the target). As discussed above, the relative positions between the markers 40 and the relative positions between a target isocenter T of the target 2 and the markers 40 can be determined with respect to an external reference frame defined by a CT scanner or other type of imaging system during a treatment planning stage before the patient is placed on the table. In the particular embodiment of the system 1 illustrated in FIGS. 8 and 9, the localization system 10 tracks the three-dimensional coordinates of the markers 40 in real time relative to an absolute external reference frame during the patient setup process and while irradiating the patient to mitigate collateral effects on adjacent healthy tissue and to ensure that the desired dosage is applied to the target.

### D. General Aspects of Markers and Localization Systems

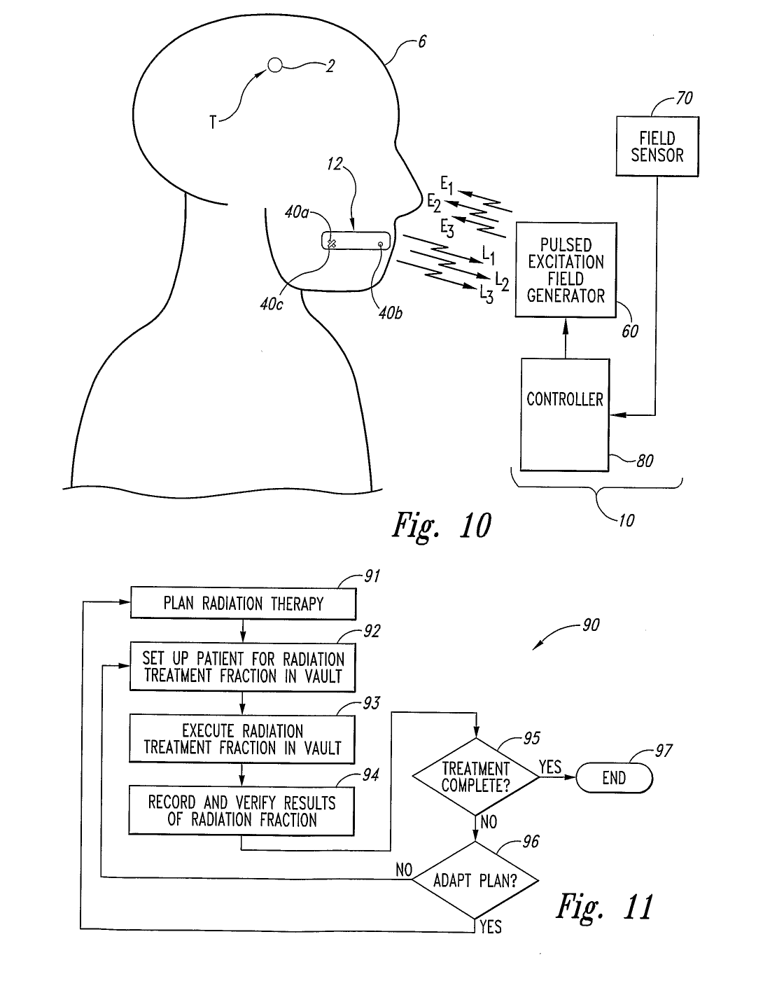

FIG. 10 is a schematic view illustrating the operation of an embodiment of the localization system 10 and markers 40a-c for treating a tumor or other target in the patient. The localization system 10 and the markers 40a-c are used to determine the location of the target 2 (FIGS. 8 and 9) before, during, and after radiation sessions. More specifically, the localization system 10 determines the locations of the markers 40a-c and provides objective target position data to a memory, user interface, linear accelerator, and/or other device in real time during setup, treatment, deployment, simulation, surgery, and/or other medical procedures.

As shown in FIG. 9, the localization system 10 may further include a patient support or alignment device 72 shown as a cradle for supporting the patient's head. The alignment device can further be a custom alignment device conformed to a specific patient's head as described in U.S. Pat. No. 5,531,229 issued on Jul. 2, 1996, hereby incorporated by reference in its entirety. An expected advantage of the localization system 10 is the elimination of an external fixation or immobilization device such as a thermoplastic mask; however, according to further embodiments, the localization system may be used in conjunction with a thermoplastic mask such as the thermoplastic mask and bite block described in U.S. Pat. No. 6,945,251 issued on Sep. 20, 2005, hereby incorporated by reference in its entirety.

In one embodiment of the localization system, real time means that indicia of objective coordinates are provided to a user interface at (a) a sufficiently high refresh rate (i.e., frequency) such that pauses in the data are not humanly discernable and (b) a sufficiently low latency to be at least substantially contemporaneous with the measurement of the location signal. In other embodiments, real time is defined by higher frequency ranges and lower latency ranges for providing the objective data to a radiation delivery device, or in still other embodiments real time is defined as providing objective data responsive to the location of the markers (e.g., at a frequency that adequately tracks the location of the target in real time and/or a latency that is substantially contemporaneous with obtaining position data of the markers).

1. Localization Systems

The localization system 10 includes an excitation source 60 (e.g., a pulsed magnetic field generator), a sensor assembly 70, and a controller 80 coupled to both the excitation source 60 and the sensor assembly 70. The excitation source 60 generates an excitation energy to energize at least one of the markers 40a-c in the patient 6 (FIG. 8). The embodiment of the excitation source 60 shown in FIG. 10 produces a pulsed magnetic field at different frequencies. For example, the excitation source 60 can frequency multiplex the magnetic field at a first frequency E1 to energize the first marker 40a, a second frequency E2 to energize the second marker 40b, and a third frequency E3 to energize the third marker 40c. In response to the excitation energy, the markers 40a-c generate location signals L1-3 at unique response frequencies. More specifically, the first marker 40a generates a first location signal L1 at a first frequency in response to the excitation energy at the first frequency E1, the second marker 40b generates a second location signal L2 at a second frequency in response to the excitation energy at the second frequency E2, and the third marker 40c generates a third location signal L3 at a third frequency in response to the excitation energy at the third frequency E3. In an alternative embodiment with two markers, the excitation source generates the magnetic field at frequencies E1 and E2, and the markers 40a-b generate location signals L1 and L2, respectively.

The sensor assembly 70 can include a plurality of coils to sense the location signals L1-3 from the markers 40a-c. The sensor assembly 70 can be a flat panel having a plurality of coils that are at least substantially coplanar relative to each other. In other embodiments, the sensor assembly 70 may be a non-planar array of coils.

The controller 80 includes hardware, software, or other computer-operable media containing instructions that operate the excitation source 60 to multiplex the excitation energy at the different frequencies E1-3. For example, the controller 80 causes the excitation source 60 to generate the excitation energy at the first frequency E1 for a first excitation period, and then the controller 80 causes the excitation source 60 to terminate the excitation energy at the first frequency E1 for a first sensing phase during which the sensor assembly 70 senses the first location signal L1 from the first marker 40a without the presence of the excitation energy at the first frequency E1. The controller 80 then causes the excitation source 60 to: (a) generate the second excitation energy at the second frequency E2 for a second excitation period; and (b) terminate the excitation energy at the second frequency E2 for a second sensing phase during which the sensor assembly 70 senses the second location signal L2 from the second marker 40b without the presence of the second excitation energy at the second frequency E2. The controller 80 then repeats this operation with the third excitation energy at the third frequency E3 such that the third marker 40c transmits the third location signal L3 to the sensor assembly 70 during a third sensing phase. As such, the excitation source 60 wirelessly transmits the excitation energy in the form of pulsed magnetic fields at the resonant frequencies of the markers 40a-c during excitation periods, and the markers 40a-c wirelessly transmit the location signals L1-3 to the sensor assembly 70 during sensing phases. It will be appreciated that the excitation and sensing phases can be repeated to permit averaging of the sensed signals to reduce noise.

The computer-operable media in the controller 80, or in a separate signal processor, or other computer also includes instructions to determine the absolute positions of each of the markers 40a-c in a three-dimensional reference frame. Based on signals provided by the sensor assembly 70 that correspond to the magnitude of each of the location signals L1-3, the controller 80 and/or a separate signal processor calculates the absolute coordinates of each of the markers 40a-c in the three-dimensional reference frame. The absolute coordinates of the markers 40a-c are objective data that can be used to calculate the coordinates of the target in the reference frame. When multiple markers are used, the rotation of the target can also be calculated.

2. Real-time Tracking

The localization system 10 and at least one marker 40 enable real-time tracking of the target 2 relative to the machine isocenter or another external reference frame outside of the patient during treatment planning, setup, radiation sessions, and at other times of the radiation therapy process. In many embodiments, real-time tracking means collecting position data of the markers, determining the locations of the markers in an external reference frame, and providing an objective output in the external reference frame that is responsive to the location of the markers. The objective output is provided at a frequency that adequately tracks the target in real time and/or a latency that is at least substantially contemporaneous with collecting the position data (e.g., within a generally concurrent period of time).

For example, several embodiments of real-time tracking are defined as determining the locations of the markers and calculating the location of the target relative to the machine isocenter at (a) a sufficiently high frequency so that pauses in representations of the target location at a user interface do not interrupt the procedure or are readily discernable by a human, and (b) a sufficiently low latency to be at least substantially contemporaneous with the measurement of the location signals from the markers. Alternatively, real time means that the localization system 10 calculates the absolute position of each individual marker 40 and/or the location of the target at a periodicity of 1 ms to 5 seconds, or in many applications at a periodicity of approximately 10-100 ms, or in some specific applications at a periodicity of approximately 20-50 ms. In applications for user interfaces, for example, the periodicity can be 12.5 ms (i.e., a frequency of 80 Hz), 16.667 ms (60 Hz), 20 ms (50 Hz), and/or 50 ms (20 Hz).

Alternatively, real-time tracking can further mean that the localization system 10 provides the absolute locations of the markers 40 and/or the target 2 to a memory device, user interface, linear accelerator, or other device within a latency of 10 ms to 5 seconds from the time the localization signals were transmitted from the markers 40. In more specific applications, the localization system generally provides the locations of the markers 40 and/or target 2 within a latency of about 20-50 ms. The localization system 10 accordingly provides real-time tracking to monitor the position of the markers 40 and/or the target 2 with respect to an external reference frame in a manner that is expected to enhance the efficacy of radiation therapy because higher radiation doses can be applied to the target and collateral effects to healthy tissue can be mitigated.

The system described herein uses one or more markers to serve as registration points to characterize target location, rotation, and motion. In accordance with aspects of the invention, the markers have a substantially fixed relationship with the target. If the markers did not have a substantially fixed relationship with the target, another type of tracking error would be incurred. This generally requires the markers to be fixed or positioned sufficiently close to the target in order that tracking errors be within clinically meaningful limits; thus, the markers may be placed in tissue or bone that exhibits representative motion of the target. For example, with respect to the head and neck, a device that is representative of the target's motion would include a mouthpiece fixedly retained in an oral cavity of a patient.

According to aspects of the present invention, the marker motion is a surrogate for the motion of the target. Accordingly, the marker is placed such that it moves in direct correlation to the target being tracked. Depending on the target being tracked, the direct correlation relationship between the target and the marker will vary. For example, with respect to soft tissue that moves substantially in response to the bony anatomy, such as the head and neck, the marker may be placed in a bite block to provide surrogate motion in direct correlation with target motion.

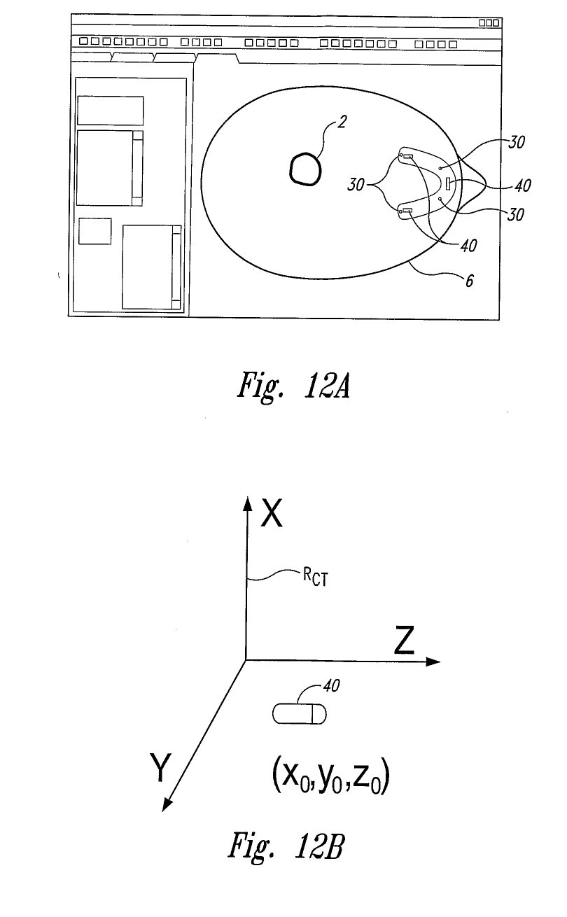

FIG. 11 is a flow diagram illustrating several aspects and uses of real-time tracking to monitor the location and the status of the target. In this embodiment, an integrated method 90 for radiation therapy includes a radiation planning procedure 91 that determines the plan for applying the radiation to the patient over a number of radiation fractions. The radiation planning procedure 91 typically includes an imaging stage in which images of a tumor or other types of targets are obtained using X-rays, CT, MR, or ultrasound imaging. The images are analyzed by a person to measure the relative distances between the markers and the relative position between the target and the markers. FIG. 12A, for example, is a representation of a CT image showing a cross-section of the patient 6, the target 2, and a marker 40. Referring to FIG. 12B, the coordinates (x₀, y₀, z₀) of the marker 40 in a reference frame RCT of the CT scanner can be determined by an operator. The coordinates of the tumor can be determined in a similar manner to ascertain the offset between the marker and the target. Alternatively, the coordinates of a radiographic fiducial 30 in a reference frame RCT of the CT scanner can be determined by an operator.

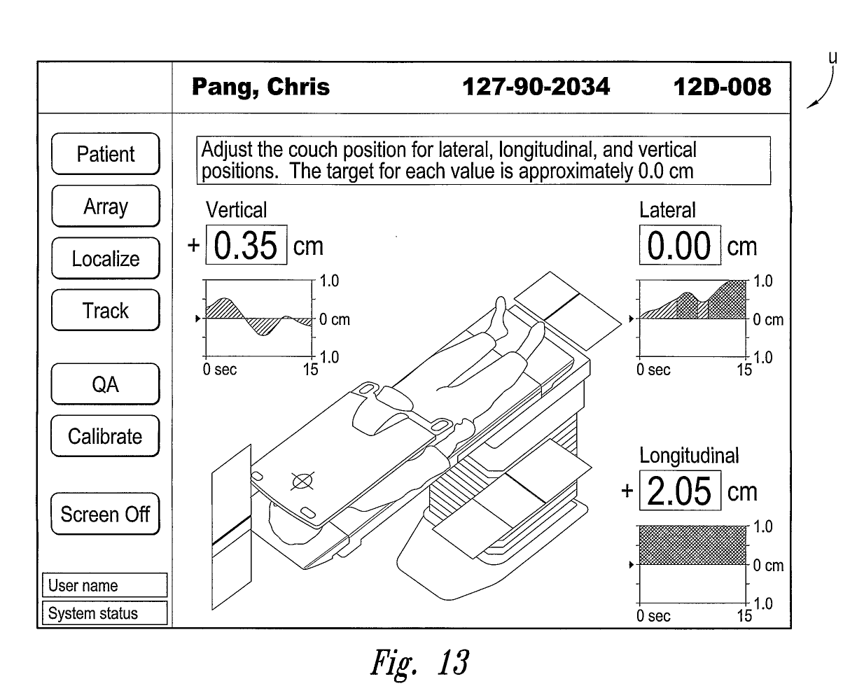

The localization system 10 and the markers 40 enable an automated patient setup process for delivering the radiation. After developing a treatment plan, the method 90 includes a setup procedure 92 in which the patient is positioned on a movable support table so that the target and markers are generally adjacent to the sensor assembly. As described above, the excitation source is activated to energize the markers, and the sensors measure the strength of the signals from the markers. The computer controller then (a) calculates objective values of the locations of the markers and the target relative to the machine isocenter, and (b) determines an objective offset value between the position of the target and the machine isocenter. Referring to FIG. 13, for example, the objective offset values can be provided to a user interface that displays the vertical, lateral, and longitudinal offsets of the target relative to the machine isocenter. A user interface may, additionally or instead, display target rotation.

One aspect of several embodiments of the localization system 10 is that the objective values are provided to the user interface or other device by processing the position data from the field sensor 70 in the controller 80 or other computer without human interpretation of the data received by the sensor assembly 70. If the offset value is outside of an acceptable range, the computer automatically activates the control system of the support table to move the tabletop relative to the machine isocenter until the target isocenter is coincident with the machine isocenter. The computer controller generally provides the objective output data of the offset to the table control system in real time as defined above. For example, because the output is provided to the radiation delivery device, it can be at a high rate (1-20 ms) and a low latency (10-20 ms). If the output data is provided to a user interface in addition to or in lieu of the table controller, it can be at a relatively lower rate (20-50 ms) and higher latency (50-200 ms).

In one embodiment, the computer controller also determines the position and orientation of the markers relative to the position and orientation of simulated markers. The locations of the simulated markers are selected so that the target will be at the machine isocenter when the real markers are at the selected locations for the simulated markers. If the markers are not properly aligned and oriented with the simulated markers, the support table is adjusted as needed for proper marker alignment. This marker alignment properly positions the target along six dimensions, namely X, Y, Z, pitch, yaw, and roll. Accordingly, the patient is automatically positioned in the correct position and rotation relative to the machine isocenter for precise delivery of radiation therapy to the target.

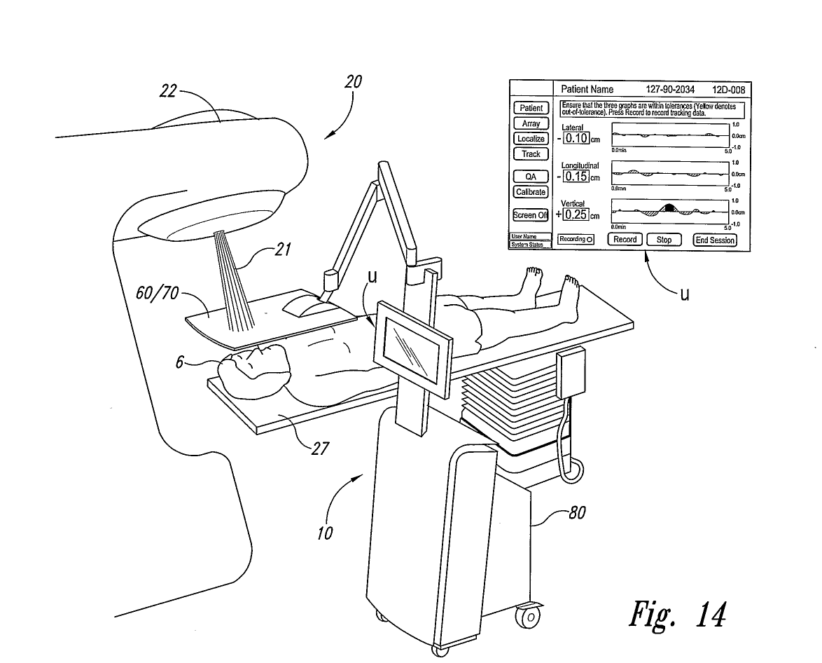

Referring back to FIG. 11, the method 90 further includes a radiation session 93. FIG. 14 shows a further aspect of an automated process in which the localization system 10 tracks the target during the radiation session 93 and controls the radiation delivery source 20 according to the offset between the target and the machine isocenter. For example, if the position of the target is outside of a permitted degree or range of displacement from the machine isocenter, the localization system 10 sends a signal to interrupt the delivery of the radiation or prevent initial activation of the beam. In another embodiment, the localization system 10 sends signals to automatically reposition a table 27 and the patient 6 (as a unit) so that the target isocenter remains within a desired range of the machine isocenter during the radiation session 93 even if the target moves. In still another embodiment, the localization system 10 sends signals to activate the radiation only when the target is within a desired range of the machine isocenter (e.g., gated therapy). In some embodiments, the localization system enables dynamic adjustment of the table 27 and/or the beam 21 in real time while irradiating the patient. Dynamic adjustment of the table 27 ensures that the radiation is accurately delivered to the target without requiring a large margin around the target.

The localization system 10 provides the objective data of the offset and/or rotation to the linear accelerator and/or the patient support table in real time as defined above. For example, as explained above with respect to automatically positioning the patent support table during the setup procedure 92, the localization system generally provides the objective output to the radiation delivery device at least substantially contemporaneously with obtaining the position data of the markers and/or at a sufficient frequency to track the target in real time. The objective output, for example, can be provided at a short periodicity (1-20 ms) and a low latency (10-20 ms) such that signals for controlling the beam 21 can be sent to the radiation delivery source 20 in the same time periods during a radiation session. In another example of real-time tracking, the objective output is provided a plurality of times during an “on-beam” period (e.g., 2, 5, 10, or more times while the beam is on). In the case of terminating or activating the radiation beam, or adjusting the leaves of a beam collimator, it is generally desirable to maximize the refresh rate and minimize the latency. In some embodiments, therefore, the localization system may provide the objective output data of the target location and/or the marker locations at a periodicity of 10 ms or less and a latency of 10 ms or less. The method 90 may further include a verification procedure 94 in which objective output data from the radiation session 93 is compared to the status of the parameters of the radiation beam.

The method 90 can further include a first decision (Block 95) in which the data from the verification procedure 94 is analyzed to determine whether the treatment is complete. If the treatment is not complete, the method 90 further includes a second decision (Block 96) in which the results of the verification procedure are analyzed to determine whether the treatment plan should be revised to compensate for changes in the target. If revisions are necessary, the method can proceed with repeating the planning procedure 91. On the other hand, if the treatment plan is providing adequate results, the method 90 can proceed by repeating the setup procedure 92, radiation session 93, and verification procedure 94 in a subsequent fraction of the radiation therapy.

The localization system 10 provides several features, either individually or in combination with each other, that enhance the ability to accurately deliver high doses of radiation to targets within tight margins. For example, many embodiments of the localization system use leadless markers that are substantially fixed with respect to the target. The markers accordingly move either directly with the target or in a relationship proportional to the movement of the target. Moreover, many aspects of the localization system 10 use a non-ionizing energy to track the leadless markers in an external, absolute reference frame in a manner that provides objective output. In general, the objective output is determined in a computer system without having a human interpret data (e.g., images) while the localization system 10 tracks the target and provides the objective output. This significantly reduces the latency between the time when the position of the marker is sensed and the objective output is provided to a device or a user. For example, this enables an objective output responsive to the location of the target to be provided at least substantially contemporaneously with collecting the position data of the marker. The system also effectively eliminates inter-user variability associated with subjective interpretation of data (e.g., images).

### E. Specific Embodiments of Markers and Localization Systems

The following specific embodiments of markers, excitation sources, sensors, and controllers provide additional details to implement the systems and processes described above with reference to FIGS. 8-14. The present inventors overcame many challenges to develop markers and localization systems that accurately determine the location of a marker which (a) produces a wirelessly transmitted location signal in response to a wirelessly transmitted excitation energy, and (b) has a cross-section small enough to be incorporated into a mouthpiece. Systems with these characteristics have several practical advantages, including (a) not requiring ionization radiation, (b) not requiring line-of-sight between the markers and sensors, and (c) effecting an objective measurement of a target's location and/or rotation. The following specific embodiments are described in sufficient detail to enable a person skilled in the art to make and use such a localization system for radiation therapy involving a tumor in the patient, but the invention is not limited to the following embodiments of markers, excitation sources, sensor assemblies, and/or controllers.

1. Markers

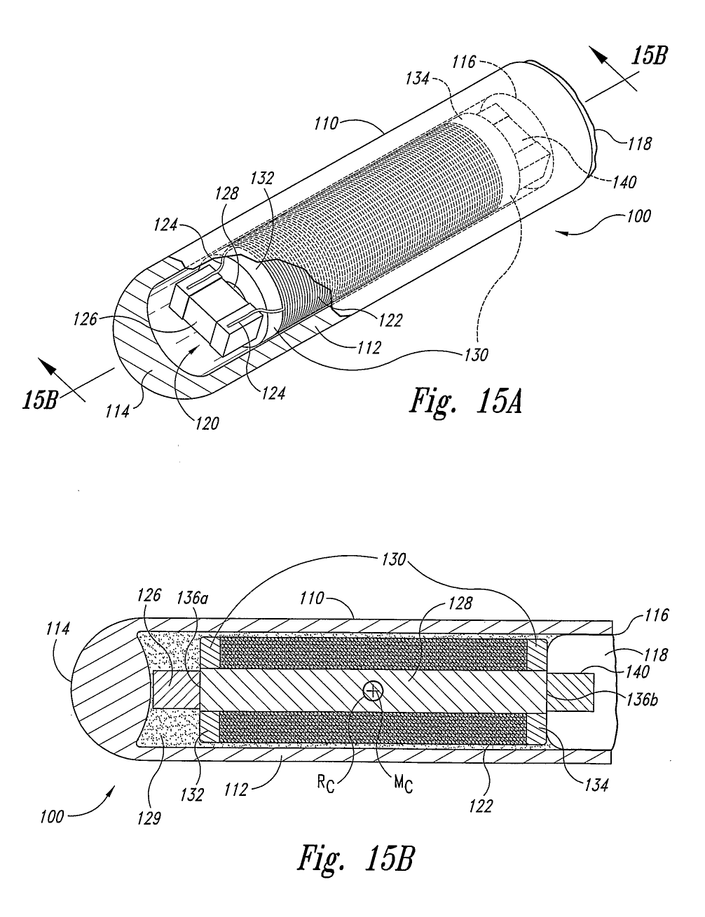

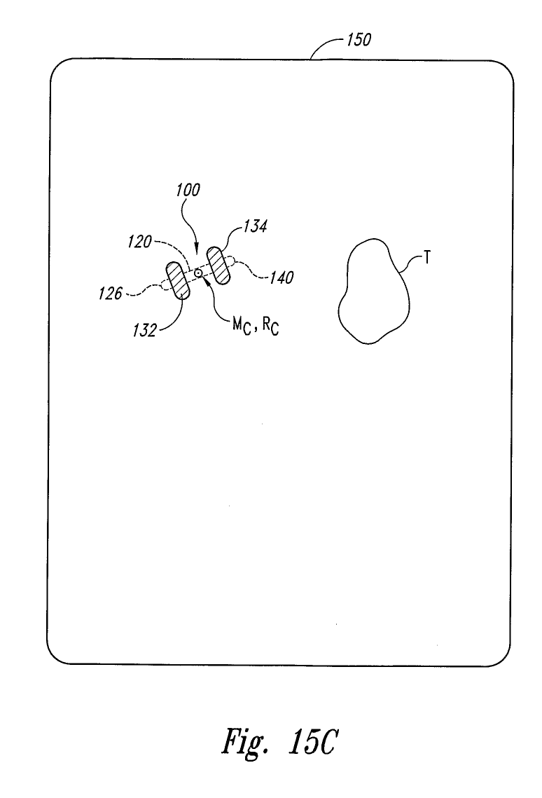

FIG. 15A is an isometric view of a marker 100 for use with the localization system 10 (FIGS. 8-14). The embodiment of the marker 100 shown in FIG. 15A includes a casing 110 and a magnetic transponder 120 (e.g., a resonating circuit) in the casing 110. The casing 110 is a barrier configured to be encased within the mouthpiece body or other instrument. The casing 110 can alternatively be configured to be adhered externally to the mouthpiece body. In one embodiment, the casing 110 includes (a) a capsule or shell 112 having a closed end 114 and an open end 116, and (b) a sealant 118 in the open end 116 of the shell 112. The casing 110 and the sealant 118 can be made from plastics, ceramics, glass, or other suitable biocompatible materials.

The magnetic transponder 120 can include a resonating circuit that wirelessly transmits a location signal in response to a wirelessly transmitted excitation field as described above. In this embodiment, the magnetic transponder 120 comprises a coil 122 defined by a plurality of windings of a conductor 124. Many embodiments of the magnetic transponder 120 also include a capacitor 126 coupled to the coil 122. The coil 122 resonates at a selected resonant frequency. The coil 122 can resonate at a resonant frequency solely using the parasitic capacitance of the windings without having a capacitor, or the resonant frequency can be produced using the combination of the coil 122 and the capacitor 126. The coil 122 accordingly generates an alternating magnetic field at the selected resonant frequency in response to the excitation energy either by itself or in combination with the capacitor 126. The conductor 124 of the illustrated embodiment can be hot air or alcohol bonded wire having a gauge of approximately 45-52. The coil 122 can have 800-1000 turns, and the windings are preferably wound in a tightly layered coil. The magnetic transponder 120 can further include a core 128 composed of a material having a suitable magnetic permeability. For example, the core 128 can be a ferromagnetic element composed of ferrite or another material. The magnetic transponder 120 can be secured to the casing 110 by an adhesive.

The marker 100 also includes an imaging element that enhances the radiographic image of the marker to make the marker more discernible in radiographic images. The imaging element also has a radiographic profile in a radiographic image such that the marker has a radiographic centroid at least approximately coincident with the magnetic centroid of the magnetic transponder 120. As explained in more detail below, the radiographic and magnetic centroids do not need to be exactly coincident with each other, but rather can be within an acceptable range. In alternative embodiments, radiographic fiducials are placed in or on the mouthpiece body in addition to the magnetic transponders.

FIG. 15B is a cross-sectional view of the marker 100 along line 15B-15B of FIG. 15A that illustrates an imaging element 130 in accordance with an embodiment of the invention. The imaging element 130 illustrated in FIGS. 15A-B includes a first contrast element 132 and second contrast element 134. The first and second contrast elements 132 and 134 are generally configured with respect to the magnetic transponder 120 so that the marker 100 has a radiographic centroid Rc that is at least substantially coincident with the magnetic centroid Mc of the magnetic transponder 120. For example, when the imaging element 130 includes two contrast elements, the contrast elements can be arranged symmetrically with respect to the magnetic transponder 120 and/or each other. The contrast elements can also be radiographically distinct from the magnetic transponder 120. In such an embodiment, the symmetrical arrangement of distinct contrast elements enhances the ability to accurately determine the radiographic centroid of the marker 100 in a radiographic image.

The first and second contrast elements 132 and 134 illustrated in FIGS. 15A-B are continuous rings positioned at opposing ends of the core 128. The first contrast element 132 can be at or around a first end 136a of the core 128, and the second contrast element 134 can be at or around a second end 136b of the core 128. The continuous rings shown in FIGS. 15A-B have substantially the same diameter and thickness. The first and second contrast elements 132 and 134, however, can have other configurations and/or be in other locations relative to the core 128 in other embodiments. For example, the first and second contrast elements 132 and 134 can be rings with different diameters and/or thicknesses. Alternatively, radiographic fiducials are distinct from the magnetic transponder such that the magnetic transponder does not contain an imaging element.

The imaging element 130, or alternatively, the radiographic fiducial, can be made from a material and configured appropriately to absorb a high fraction of incident photons of a radiation beam used for producing the radiographic image. For example, when the imaging radiation has high acceleration voltages in the megavoltage range, the imaging element 130, or radiographic fiducial, is made from, at least in part, high-density materials with sufficient thickness and cross-sectional area to absorb enough of the photon fluence incident on the imaging element to be visible in the resulting radiograph. Many high energy beams used for therapy have acceleration voltages of 6 MV-25 MV, and these beams are often used to produce radiographic images in the 5 MV-10 MV range, or more specifically in the 6 MV-8 MV range. As such, the imaging element 130, or radiographic fiducial, can be made from a material that is sufficiently absorbent of incident photon fluence to be visible in an image produced using a beam with an acceleration voltage of 5 MV-10 MV, or more specifically an acceleration voltage of 6 MV-8 MV.

Several specific embodiments of imaging elements 130, or radiographic fiducials, can be made from gold, tungsten, platinum and/or other high-density metals. In these embodiments the imaging element 130, or radiographic fiducial, can be composed of materials having a density of 19.25 g/cm³ (density of tungsten) and/or a density of approximately 21.4 g/cm³ (density of platinum). Many embodiments of the imaging element 130, or radiographic fiducial, accordingly have a density not less than 19 g/cm³. In other embodiments, however, the material(s) of the imaging element 130, or radiographic fiducial, can have a substantially lower density. For example, imaging elements with lower density materials are suitable for applications that use lower energy radiation to produce radiographic images. Moreover, with respect to the imaging element 130, the first and second contrast elements 132 and 134 can be composed of different materials such that the first contrast element 132 can be made from a first material and the second contrast element 134 can be made from a second material.

Referring to FIG. 15B, the marker 100 can further include a module 140 at an opposite end of the core 128 from the capacitor 126. In the embodiment of the marker 100 shown in FIG. 15B, the module 140 is configured to be symmetrical with respect to the capacitor 126 to enhance the symmetry of the radiographic image. As with the first and second contrast elements 132 and 134, the module 140 and the capacitor 126 are arranged such that the magnetic centroid of the marker is at least approximately coincident with the radiographic centroid of the marker 100. The module 140 can be another capacitor that is identical to the capacitor 126, or the module 140 can be an electrically inactive element. Suitable electrically inactive modules include ceramic blocks shaped like the capacitor 126 and located with respect to the coil 122, the core 128, and the imaging element 130 to be symmetrical with each other. In still other embodiments the module 140 can be a different type of electrically active element electrically coupled to the magnetic transponder 120.

One specific process of using the marker involves imaging the marker using a first modality and then tracking the target of the patient and/or the marker using a second modality. For example, the location of the marker relative to the target can be determined by imaging the marker and the target using radiation. The marker and/or the target can then be localized and tracked using the magnetic field generated by the marker in response to an excitation energy. Alternatively, the body may include a transponder and a radiographic fiducial such that another specific process of using the marker involves imaging the fiducial using a first modality and then tracking the transponder and/or the target of the patient using a second modality.

The marker 100 shown in FIGS. 15A-B is expected to provide an enhanced radiographic image compared to conventional magnetic markers and is useful for more accurately determining the relative position between the marker and the target of a patient. FIG. 15C, for example, illustrates a radiographic image 150 of the marker 100 and a target T of the patient. The first and second contrast elements 132 and 134 are expected to be more distinct in the radiographic image 150 because they can be composed of higher density materials than the components of the magnetic transponder 120. The first and second contrast elements 132 and 134 can accordingly appear as bulbous ends of a dumbbell shape in applications in which the components of the magnetic transponder 120 are visible in the image. In certain megavolt applications, the components of the magnetic transponder 120 may not appear at all on the radiographic image 150 such that the first and second contrast elements 132 and 134 will appear as distinct regions that are separate from each other. In either embodiment, the first and second contrast elements 132 and 134 provide a reference frame in which the radiographic centroid Rc of the marker 100 can be located in the image 150. Moreover, because the imaging element 130 is configured so that the radiographic centroid Rc is at least approximately coincident with the magnetic centroid Mc, the relative offset or position between the target T and the magnetic centroid Mc can be accurately determined using the marker 100. The embodiment of the marker 100 illustrated in FIGS. 15A-C, therefore, is expected to mitigate errors caused by incorrectly estimating the radiographic and magnetic centroids of markers in radiographic images.

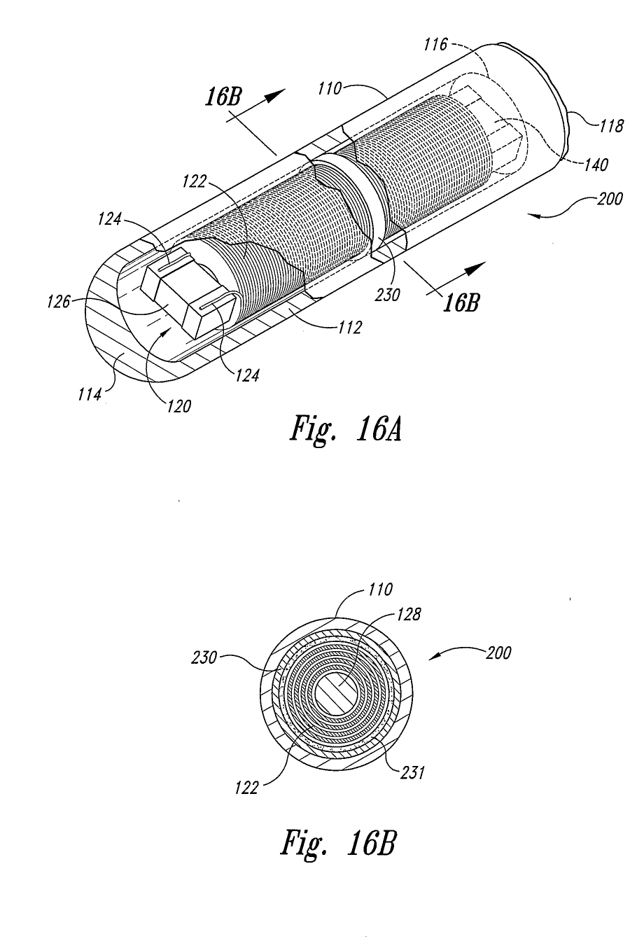

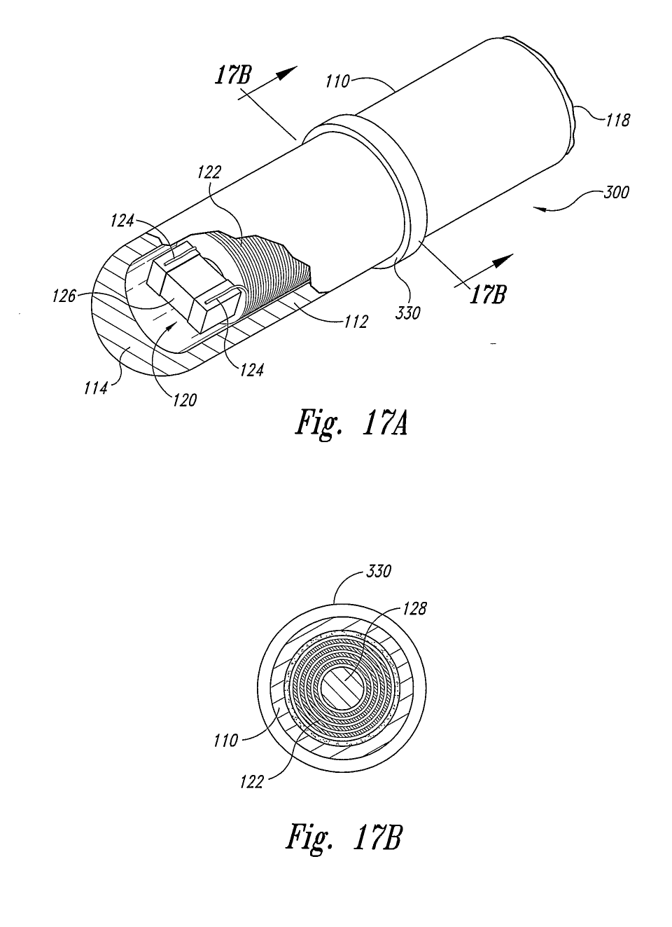

FIG. 16A is an isometric view of a marker 200 with a cut-away portion to illustrate internal components, and FIG. 16B is a cross-sectional view of the marker 200 taken along line 16B-16B of FIG. 16A. The marker 200 is similar to the marker 100 shown above in FIG. 15A, and thus like reference numbers refer to like components. The marker 200 differs from the marker 100 in that the marker 200 includes an imaging element 230 defined by a single contrast element. The imaging element 230 is generally configured relative to the magnetic transponder 120 so that the radiographic centroid of the marker 200 is at least approximately coincident with the magnetic centroid of the magnetic transponder 120. The imaging element 230, more specifically, is a ring extending around the coil 122 at a medial region of the magnetic transponder 120. The imaging element 230 can be composed of the same materials described above with respect to the imaging element 130 in FIGS. 15A-B. The imaging element 230 can have an inner diameter that is approximately equal to the outer diameter of the coil 122, and an outer diameter within the casing 110. As shown in FIG. 16B, however, a spacer 231 can be between the inner diameter of the imaging element 230 and the outer diameter of the coil 122.

The marker 200 is expected to operate in a manner similar to the marker 100 described above. The marker 200, however, does not have two separate contrast elements that provide two distinct, separate points in a radiographic image. The imaging element 230 is still highly useful in that it identifies the radiographic centroid of the marker 200 in a radiographic image, and it can be configured so that the radiographic centroid of the marker 200 is at least approximately coincident with the magnetic centroid of the magnetic transponder 120.

FIG. 17A is an isometric view of a marker 300 having a cut-away portion, and FIG. 17B is a cross-sectional view of the marker 300 taken along line 17B-17B of FIG. 17A. The marker 300 is substantially similar to the marker 200 shown in FIGS. 16A-B, and thus like reference numbers refer to like components in FIGS. 15A-17B. The imaging element 330 can be a high-density ring configured relative to the magnetic transponder 120 so that the radiographic centroid of the marker 300 is at least approximately coincident with the magnetic centroid of the magnetic transponder 120. The marker 300, more specifically, includes an imaging element 330 around the casing 110. The marker 300 is expected to operate in much the same manner as the marker 200 shown in FIGS. 16A-B.

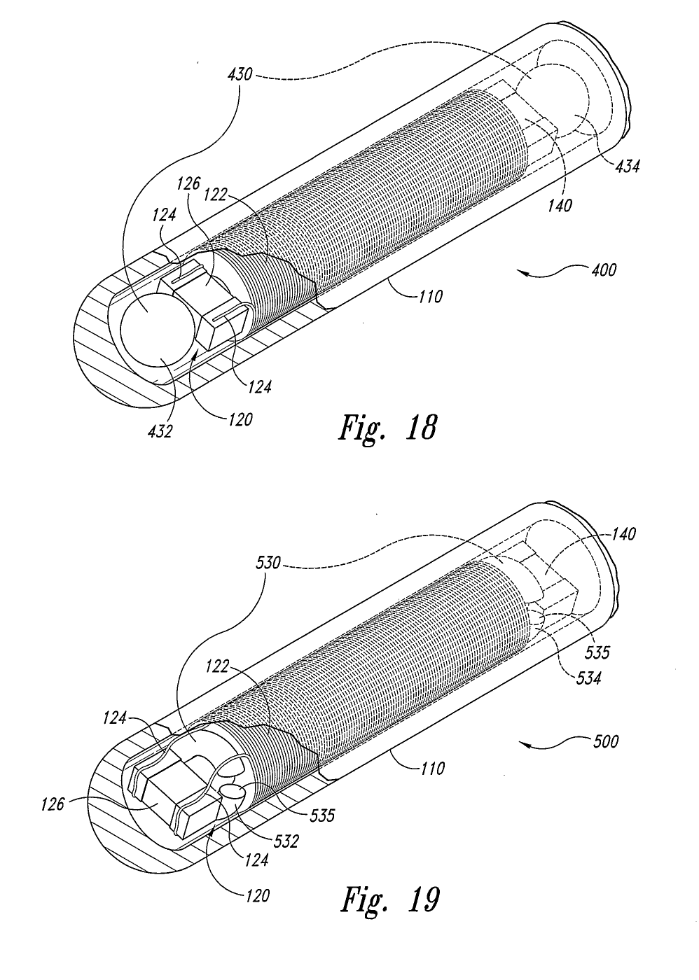

FIG. 18 is an isometric view with a cut-away portion illustrating a marker 400 in accordance with another embodiment of the invention. The marker 400 is similar to the marker 100 shown in FIGS. 15A-C, and thus like reference numbers refer to like components in these Figures. The marker 400 has an imaging element 430 including a first contrast element 432 at one end of the magnetic transponder 120 and a second contrast element 434 at another end of the magnetic transponder 120. The first and second contrast elements 432 and 434 are spheres composed of suitable high-density materials. The contrast elements 432 and 434, for example, can be composed of gold, tungsten, platinum, or other suitable high-density materials for use in radiographic imaging. The marker 400 is expected to operate in a manner similar to the marker 100, as described above.

FIG. 19 is an isometric view with a cut-away portion of a marker 500 in accordance with yet another embodiment of the invention. The marker 500 is substantially similar to the markers 100 and 400 shown in FIGS. 15A and 18, and thus like reference numbers refer to like components in these Figures. The marker 500 includes an imaging element 530 including a first contrast element 532 and a second contrast element 534. The first and second contrast elements 532 and 534 can be positioned proximate to opposing ends of the magnetic transponder 120. The first and second contrast elements 532 and 534 can be discontinuous rings having a gap 535 to mitigate eddy currents. The contrast elements 532 and 534 can be composed of the same materials as described above with respect to the contrast elements of other imaging elements in accordance with other embodiments of the invention.

Additional embodiments of markers in accordance with the invention can include imaging elements incorporated into or otherwise integrated with the casing 110, the core 128 (FIG. 15B) of the magnetic transponder 120, and/or the adhesive 129 (FIG. 15B) in the casing. For example, particles of a high-density material can be mixed with ferrite and extruded to form the core 128. Alternative embodiments can mix particles of a high-density material with glass or another material to form the casing 110, or coat the casing 110 with a high-density material. In still other embodiments, a high-density material can be mixed with the adhesive 129 and injected into the casing 110. Any of these embodiments can incorporate the high-density material into a combination of the casing 110, the core 128 and/or the adhesive 129. Suitable high-density materials can include tungsten, gold, and/or platinum as described above. In still other embodiments, the radiographic fiducial element may be distinct from the transponder. In still other embodiments, the transponder may be encased in the mouthpiece body such that a separate casing 110 is not required.

The markers described above with reference to FIGS. 15A-19 can be used for the markers 40 in the localization system 10 (FIGS. 1-14). The localization system 10 can have several markers with the same type of imaging elements, or markers with different imaging elements can be used with the same instrument. Several additional details of these markers and other embodiments of markers are described in U.S. application Ser. Nos. 10/334,698 and 10/746,888, which are incorporated herein by reference. For example, the markers may not have any imaging elements for applications with lower energy radiation, or the markers may have reduced volumes of ferrite and metals to mitigate issues with MR imaging as set forth in U.S. application Ser. No. 10/334,698.

2. Localization Systems

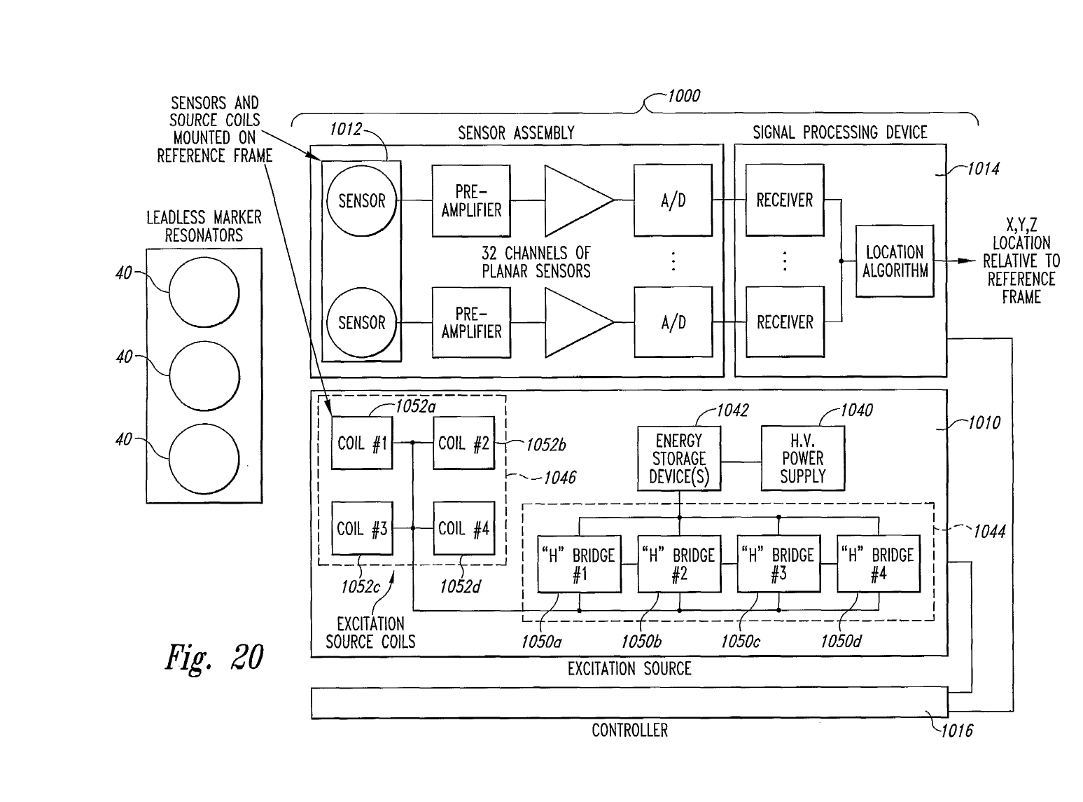

FIG. 20 is a schematic block diagram of a localization system 1000 for determining the absolute location of the markers 40 (shown schematically) relative to a reference frame. The localization system 1000 includes an excitation source 1010, a sensor assembly 1012, a signal processor 1014 operatively coupled to the sensor assembly 1012, and a controller 1016 operatively coupled to the excitation source 1010 and the signal processor 1014. The excitation source 1010 is one embodiment of the excitation source 60 described above with reference to FIG. 10; the sensor assembly 1012 is one embodiment of the sensor assembly 70 described above with reference to FIG. 10; and the controller 1016 is one embodiment of the controller 80 described above with reference to FIG. 10.

The excitation source 1010 is adjustable to generate a magnetic field having a waveform with energy at selected frequencies to match the resonant frequencies of the markers 40. The magnetic field generated by the excitation source 1010 energizes the markers 40 at their respective frequencies. After the markers 40 have been energized, the excitation source 1010 is momentarily switched to an “off” position so that the pulsed magnetic excitation field is terminated while the markers wirelessly transmit the location signals. This allows the sensor assembly 1012 to sense the location signals from the markers 40 without measurable interference from the significantly more powerful magnetic field from the excitation source 1010. The excitation source 1010 accordingly allows the sensor assembly 1012 to measure the location signals from the markers 40 at a sufficient signal-to-noise ratio so that the signal processor 1014 or the controller 1016 can accurately calculate the absolute location of the markers 40 relative to a reference frame.

a. Excitation Sources

Referring still to FIG. 20, the excitation source 1010 includes a high-voltage power supply 1040, an energy storage device 1042 coupled to the power supply 1040, and a switching network 1044 coupled to the energy storage device 1042. The excitation source 1010 also includes a coil assembly 1046 coupled to the switching network 1044. In one embodiment, the power supply 1040 is a 500-volt power supply, although other power supplies with higher or lower voltages can be used. The energy storage device 1042 in one embodiment is a high-voltage capacitor that can be charged and maintained at a relatively constant charge by the power supply 1040. The energy storage device 1042 alternately provides energy to and receives energy from the coils in the coil assembly 1046.

The energy storage device 1042 is capable of storing adequate energy to reduce voltage drop in the energy storage device while having a low series resistance to reduce power losses. The energy storage device 1042 also has a low series inductance to more effectively drive the coil assembly 1046. Suitable capacitors for the energy storage device 1042 include aluminum electrolytic capacitors used in flash energy applications. Alternative energy storage devices can also include NiCd and lead acid batteries, as well as alternative capacitor types, such as tantalum, film, or the like.

The switching network 1044 includes individual H-bridge switches 1050 (identified individually by reference numbers 1050a-d), and the coil assembly 1046 includes individual source coils 1052 (identified individually by reference numbers 1052a-d). Each H-bridge switch 1050 controls the energy flow between the energy storage device 1042 and one of the source coils 1052. For example, H-bridge switch #1 1050a independently controls the flow of the energy to/from source coil #1 1052a, H-bridge switch #2 1050b independently controls the flow of the energy to/from source coil #2 1052b, H-bridge switch #3 1050c independently controls the flow of the energy to/from source coil #3 1052c, and H-bridge switch #4 1050d independently controls the flow of the energy to/from source coil #4 1052d. The switching network 1044 accordingly controls the phase of the magnetic field generated by each of the source coils 1052a-d independently. The H-bridge switches 1050 can be configured so that the electrical signals for all the source coils 1052 are in phase, or the H-bridge switches 1050 can be configured so that one or more of the source coils 1052 are 180° out of phase. Furthermore, the H-bridge switches 1050 can be configured so that the electrical signals for one or more of the source coils 1052 are between 0° and 180° out of phase to simultaneously provide magnetic fields with different phases.

The source coils 1052 can be arranged in a coplanar array that is fixed relative to the reference frame. Each source coil 1052 can be a square, planar winding arranged to form a flat, substantially rectilinear coil. The source coils 1052 can have other shapes and other configurations in different embodiments. In one embodiment, the source coils 1052 are individual conductive lines formed in a stratum of a printed circuit board, or windings of a wire in a foam frame. Alternatively, the source coils 1052 can be formed in different substrates or arranged so that two or more of the source coils 1052 are not planar with one another. Additionally, alternate embodiments of the invention may have fewer or more source coils than illustrated in FIG. 20.

The selected magnetic fields from the source coils 1052 combine to form an adjustable excitation field that can have different three-dimensional shapes to excite the markers 40 at any spatial orientation within an excitation volume. When the planar array of the source coils 1052 is generally horizontal, the excitation volume is positioned above an area approximately corresponding to the central region of the coil assembly 1046. The excitation volume is the three-dimensional space adjacent to the coil assembly 1046 in which the strength of the magnetic field is sufficient to adequately energize the markers 40.

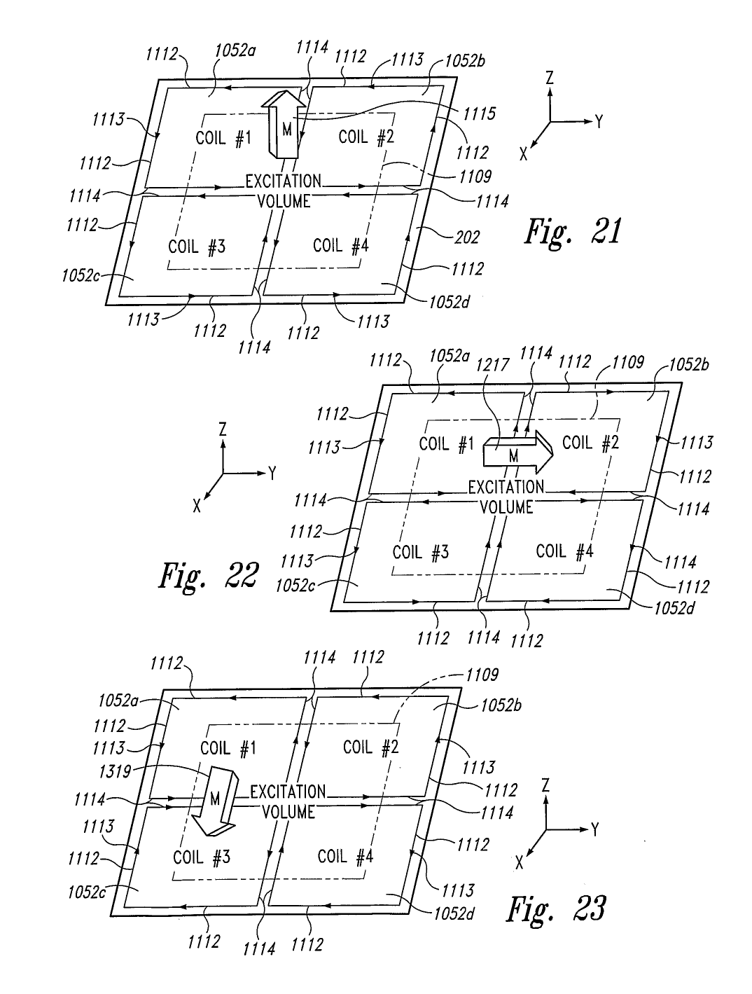

FIGS. 21-23 are schematic views of a planar array of the source coils 1052 with the alternating electrical signals provided to the source coils in different combinations of phases to generate excitation fields about different axes relative to the illustrated XYZ coordinate system. Each source coil 1052 has two outer sides 1112 and two inner sides 1114. Each inner side 1114 of one source coil 1052 is immediately adjacent to an inner side 1114 of another source coil 1052, but the outer sides 1112 of all the source coils 1052 are not adjacent to any other source coil 1052.

In the embodiment of FIG. 21, all the source coils 1052a-d simultaneously receive an alternating electrical signal in the same phase. As a result, the electrical current flows in the same direction through all the source coils 1052a-d such that a direction 1113 of the current flowing along the inner sides 1114 of one source coil (e.g., source coil 1052a) is opposite to the direction 1113 of the current flowing along the inner sides 1114 of the two adjacent source coils (e.g., source coils 1052c and 1052d). The magnetic fields generated along the inner sides 1114 accordingly cancel each other out so that the magnetic field is effectively generated from the current flowing along the outer sides 1112 of the source coils 1052a-d. The resulting excitation field formed by the combination of the magnetic fields from the source coils 1052a-d shown in FIG. 21 has a magnetic moment 1115 generally in the Z direction within an excitation volume 1109. This excitation field energizes markers parallel to the Z-axis or markers positioned with an angular component along the Z-axis (i.e., not orthogonal to the Z-axis).

FIG. 22 is a schematic view of the source coils 1052a-d with the alternating electrical signals provided in a second combination of phases to generate a second excitation field with a different spatial orientation. In this embodiment, source coils 1052a and 1052c are in phase with each other, and source coils 1052b and 1052d are in phase with each other. However, source coils 1052a and 1052c are 180° out of phase with source coils 1052b and 1052d. The magnetic fields from the source coils 1052a-d combine to generate an excitation field having a magnetic moment 1217 generally in the Y direction within the excitation volume 1109. Accordingly, this excitation field energizes markers parallel to the Y-axis or markers positioned with an angular component along the Y-axis.

FIG. 23 is a schematic view of the source coils 1052a-d with the alternating electrical signals provided in a third combination of phases to generate a third excitation field with a different spatial orientation. In this embodiment, source coils 1052a and 1052b are in phase with each other, and source coils 1052c and 1052d are in phase with each other. However, source coils 1052a and 1052b are 180° out of phase with source coils 1052c and 1052d. The magnetic fields from the source coils 1052a-d combine to generate an excitation field having a magnetic moment 1319 in the excitation volume 1109 generally in the direction of the X-axis. Accordingly, this excitation field energizes markers parallel to the X-axis or markers positioned with an angular component along the X-axis.

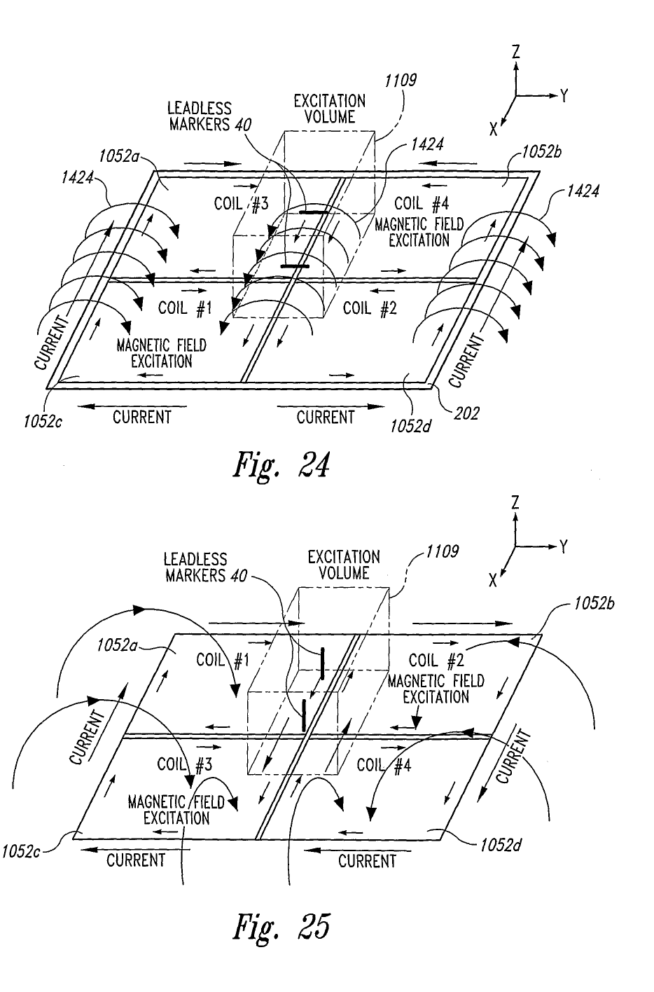

FIG. 24 is a schematic view of the source coils 1052a-d illustrating the current flow to generate an excitation field 1424 for energizing markers 40 with longitudinal axes parallel to the Y-axis. The switching network 1044 (FIG. 20) is configured so that the phases of the alternating electrical signals provided to the source coils 1052a-d are similar to the configuration of FIG. 22. This generates the excitation field 1424 with a magnetic moment in the Y direction to energize the markers 40.

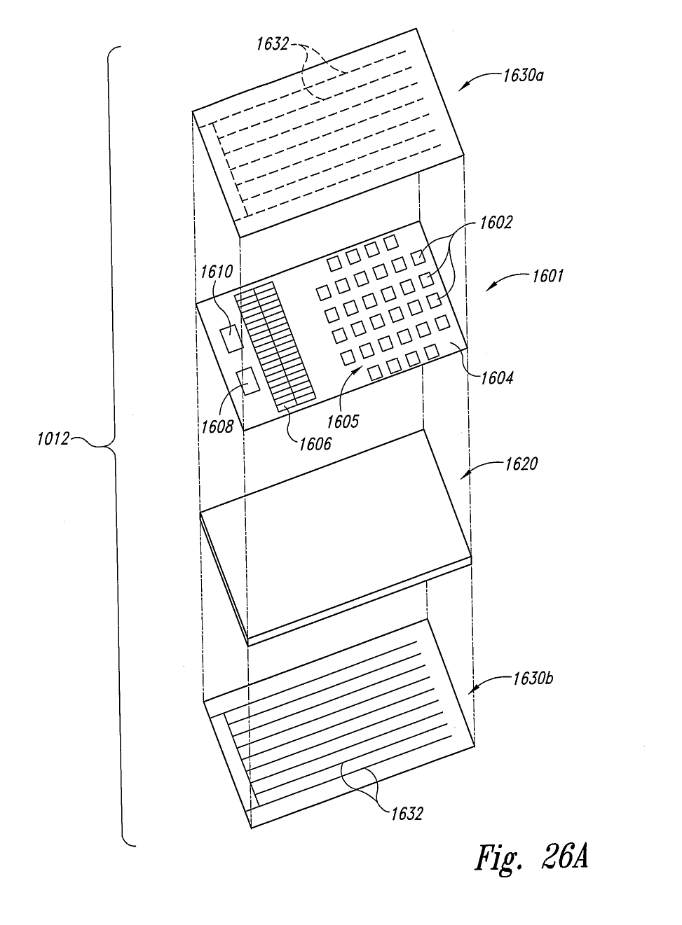

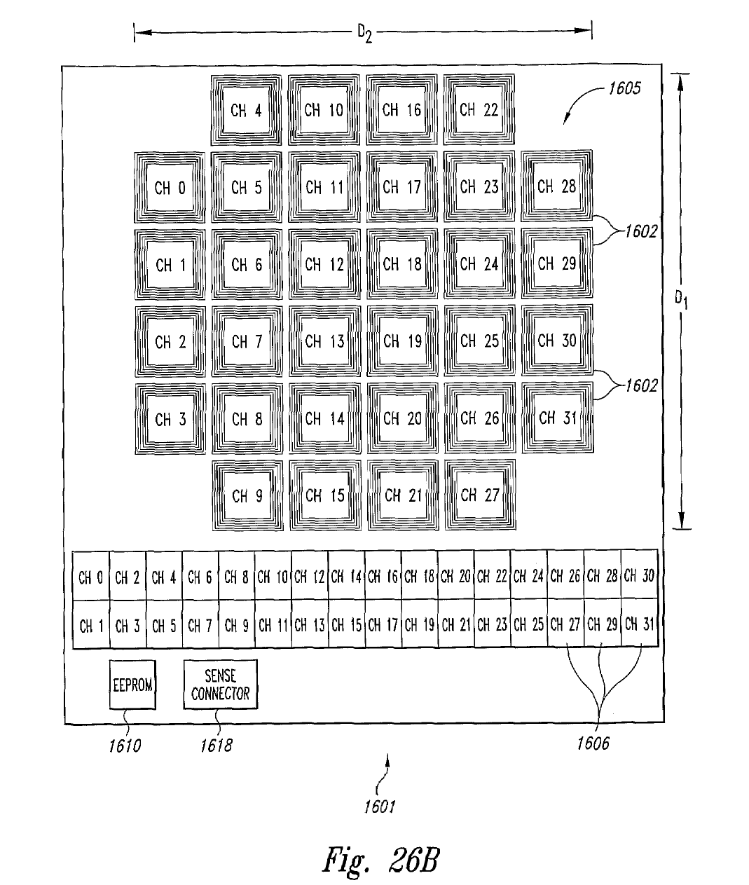

FIG. 25 further illustrates the ability to spatially adjust the excitation field in a manner that energizes any of the markers 40 at different spatial orientations. In this embodiment, the switching network 1044 (FIG. 20) is configured so that the phases of the alternating electrical signals provided to the source coils 1052a-d are similar to the configuration shown in FIG. 21. This produces an excitation field with a magnetic moment in the Z direction that energizes markers 40 with longitudinal axes parallel to the Z-axis.

The spatial configuration of the excitation field in the excitation volume 1109 can be quickly adjusted by manipulating the switching network 1044 (FIG. 20) to change the phases of the electrical signals provided to the source coils 1052a-d. As a result, the overall magnetic excitation field can be changed to be oriented in either the X, Y or Z direction within the excitation volume 1109. This adjustment of the spatial orientation of the excitation field reduces or eliminates blind spots in the excitation volume 1109. Therefore, the markers 40 within the excitation volume 1109 can be energized by the source coils 1052a-d regardless of the spatial orientations of the markers 40.

In one embodiment, the excitation source 1010 is coupled to the sensor assembly 1012 so that the switching network 1044 (FIG. 20) adjusts orientation of the pulsed generation of the excitation field along the X, Y, and Z axes depending upon the strength of the signal received by the sensor assembly. If the location signal from a marker 40 is insufficient, the switching network 1044 can automatically change the spatial orientation of the excitation field during a subsequent pulsing of the source coils 1052a-d to generate an excitation field with a moment in the direction of a different axis or between axes. The switching network 1044 can be manipulated until the sensor assembly 1012 receives a sufficient location signal from the marker 40.

The excitation source 1010 illustrated in FIG. 20 alternately energizes the source coils 1052a-d during an excitation phase to power the markers 40, and then actively de-energizes the source coils 1052a-d during a sensing phase in which the sensor assembly 1012 senses the decaying location signals wirelessly transmitted by the markers 40. To actively energize and de-energize the source coils 1052a-d, the switching network 1044 is configured to alternatively transfer stored energy from the energy storage device 1042 to the source coils 1052a-d, and to then re-transfer energy from the source coils 1052a-d back to the energy storage device 1042. The switching network 1044 alternates between first and second “on” positions so that the voltage across the source coils 1052 alternates between positive and negative polarities. For example, when the switching network 1044 is switched to the first “on” position, the energy in the energy storage device 1042 flows to the source coils 1052a-d. When the switching network 1044 is switched to the second “on” position, the polarity is reversed such that the energy in the source coils 1052a-d is actively drawn from the source coils 1052a-d and directed back to the energy storage device 1042. As a result, the energy in the source coils 1052a-d is quickly transferred back to the energy storage device 1042 to abruptly terminate the excitation field transmitted from the source coils 1052a-d and to conserve power consumed by the energy storage device 1042. This removes the excitation energy from the environment so that the sensor assembly 1012 can sense the location signals from the markers 40 without interference from the significantly larger excitation energy from the excitation source 1010. Several additional details of the excitation source 1010 and alternate embodiments are disclosed in U.S. patent application Ser. No. 10/213,980 filed on Aug. 7, 2002, and now U.S. Pat. No. 6,822,570, which is incorporated by reference herein in its entirety.

b. Sensor Assemblies

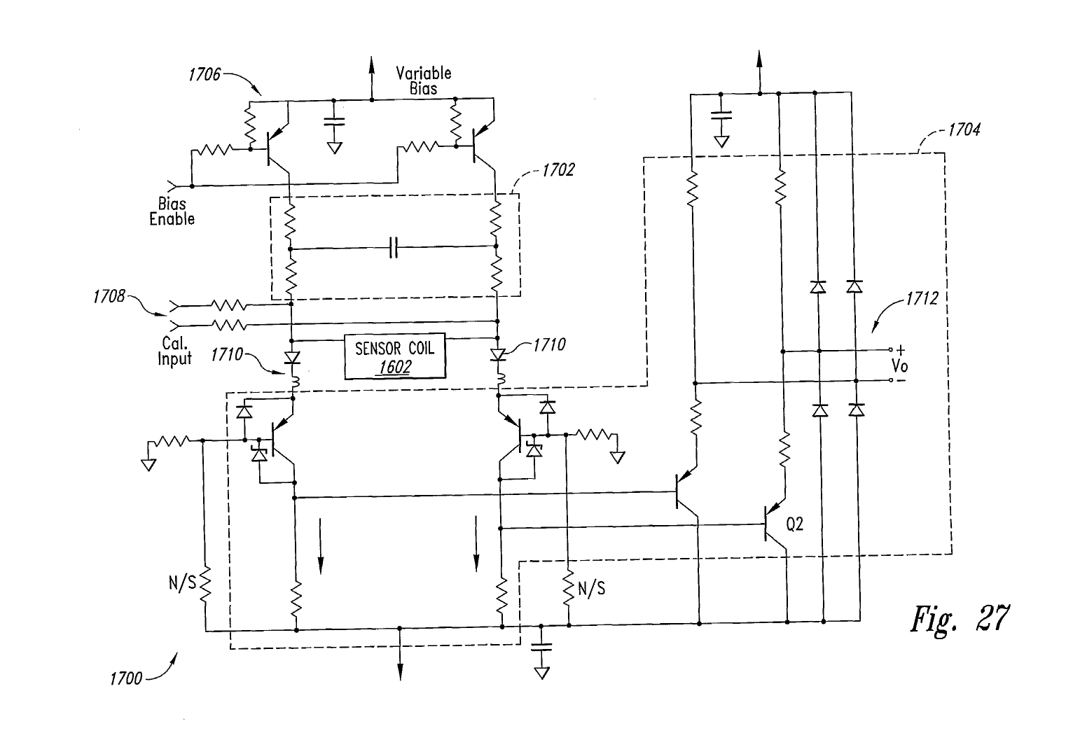

FIG. 26A is an exploded isometric view showing several components of the sensor assembly 1012 for use in the localization system 1000 (FIG. 20). The sensor assembly 1012 includes a sensing unit 1601 having a plurality of coils 1602 formed on or carried by a panel 1604. The coils 1602 can be field sensors or magnetic flux sensors arranged in a sensor array 1605.

The panel 1604 may be a substantially non-conductive material, such as a sheet of KAPTON® produced by DuPont. KAPTON® is particularly useful when an extremely stable, tough, and thin film is required (such as to avoid radiation beam contamination), but the panel 1604 may be made from other materials and have other configurations. For example, FR4 (epoxy-glass substrates), GETEK or other Teflon-based substrates, and other commercially available materials can be used for the panel 1604. Additionally, although the panel 1604 may be a flat, highly planar structure, in other embodiments, the panel 1604 may be curved along at least one axis. In either embodiment, the field sensors (e.g., coils) are arranged in a locally planar array in which the plane of one field sensor is at least substantially coplanar with the planes of adjacent field sensors. For example, the angle between the plane defined by one coil relative to the planes defined by adjacent coils can be from approximately 0° to 10°, and more generally is less than 5°. In some circumstances, however, one or more of the coils may be at an angle greater than 10° relative to other coils in the array.

The sensor assembly 1012 shown in FIG. 26A can optionally include a core 1620 laminated to the panel 1604. The core 1620 can be a support member made from a material, or the core 1620 can be a low-density foam, such as a closed-cell Rohacell foam. The core 1620 is preferably a stable layer that has a low coefficient of thermal expansion so that the shape of the sensor assembly 1012 and the relative orientation between the coils 1602 remain within a defined range over an operating temperature range.

The sensor assembly 1012 can further include a first exterior cover 1630a on one side of the sensing subsystem and a second exterior cover 1630b on an opposing side. The first and second exterior covers 1630a-b can be thin, thermally stable layers, such as Kevlar or Thermount films. Each of the first and second exterior covers 1630a-b can include electric shielding 1632 to block undesirable external electric fields from reaching the coils 1602. The electric shielding 1632 can be a plurality of parallel legs of gold-plated copper strips to define a comb-shaped shield in a configuration commonly called a Faraday shield. It will be appreciated that the shielding can be formed from other materials that are suitable for shielding. The electric shielding 1632 can be formed on the first and second exterior covers 1630a-b using printed circuit board manufacturing technology or other techniques.

The panel 1604 with the coils 1602 is laminated to the core 1620 using a pressure sensitive adhesive or another type of adhesive. The first and second exterior covers 1630a-b are similarly laminated to the assembly of the panel 1604 and the core 1620. The laminated assembly forms a rigid structure that fixedly retains the arrangement of the coils 1602 in a defined configuration over a large operating temperature range. As such, the sensor assembly 1012 does not substantially deflect across its surface during operation. The sensor assembly 1012, for example, can retain the array of coils 1602 in the fixed position with a deflection of no greater than ±0.5 mm, and in some cases no more than ±0.3 mm. The stiffness of the sensing subsystem provides very accurate and repeatable monitoring of the precise location of leadless markers in real time.

In still another embodiment, the sensor assembly 1012 can further include a plurality of source coils that are a component of the excitation source 1010. One suitable array combining the sensor assembly 1012 with source coils is disclosed in U.S. patent application Ser. No. 10/334,700, entitled PANEL-TYPE SENSOR/SOURCE ARRAY ASSEMBLY, filed on Dec. 30, 2002, which is incorporated by reference herein in its entirety.

FIG. 26B further illustrates an embodiment of the sensing unit 1601. In this embodiment, the sensing unit 1601 includes 32 coils 1602; each coil 1602 is associated with a separate channel 1606 (shown individually as channels “Ch 0” through “Ch 31”). The overall dimension of the panel 1604 can be approximately 40 cm by 54 cm, but the array 1605 has a first dimension D1 of approximately 40 cm and a second dimension D2 of approximately 40 cm. The array 1605 can have other sizes or other configurations (e.g., circular) in alternative embodiments. Additionally, the array 1605 can have more or fewer coils, such as 8-64 coils; the number of coils may moreover be a power of 2.

The coils 1602 may be conductive traces or depositions of copper or another suitably conductive metal formed on the panel 1604. Each coil 1602 has a trace with a width of approximately 0.15 mm and a spacing between adjacent turns within each coil of approximately 0.13 mm. The coils 1602 can have approximately 15 to 90 turns, and in specific applications each coil has approximately 40 turns. Coils with less than 15 turns may not be sensitive enough for some applications, and coils with more than 90 turns may lead to excessive voltage from the source signal during excitation and excessive settling times resulting from the coil's lower self-resonant frequency. In other applications, however, the coils 1602 can have less than 15 turns or more than 90 turns.

As shown in FIG. 26B, the coils 1602 are arranged as square spirals, although other configurations may be employed, such as arrays of circles, interlocking hexagons, triangles, etc. Such square spirals utilize a large percentage of the surface area to improve the signal to noise ratio. Square coils also simplify design layout and modeling of the array compared to circular coils; for example, circular coils could waste surface area for linking magnetic flux from the markers 40. The coils 1602 have an inner dimension of approximately 40 mm, and an outer dimension of approximately 62 mm, although other dimensions are possible depending upon applications. Sensitivity may be improved with an inner dimension as close to an outer dimension as possible given manufacturing tolerances. In several embodiments, the coils 1602 are identical to each other or at least configured substantially similarly.

The pitch of the coils 1602 in the array 1605 is a function of, at least in part, the minimum distance between the marker and the coil array. In one embodiment, the coils are arranged at a pitch of approximately 67 mm. This specific arrangement is particularly suitable when the wireless markers 40 are positioned approximately 7-27 cm from the sensor assembly 1012. If the wireless markers are closer than 7 cm, then the sensing subsystem may include sensor coils arranged at a smaller pitch. In general, a smaller pitch is desirable when wireless markers are to be sensed at a relatively short distance from the array of coils. The pitch of the coils 1602, for example, is approximately 50%-200% of the minimum distance between the marker and the array.

In general, the size and configuration of the array 1605 and the coils 1602 in the array depend on the frequency range in which they are to operate, the distance from the markers 40 to the array, the signal strength of the markers, and several other factors. Those skilled in the relevant art will readily recognize that other dimensions and configurations may be employed depending, at least in part, on a desired frequency range and distance from the markers to the coils.

The array 1605 is sized to provide a large aperture to measure the magnetic field emitted by the markers. It can be particularly challenging to accurately measure the signal emitted by an marker that wirelessly transmits a marker signal in response to a wirelessly transmitted energy source because the marker signal is much smaller than the source signal and other magnetic fields in a room (e.g., magnetic fields from CRTs, etc.). The size of the array 1605 can be selected to preferentially measure the near field of the marker while mitigating interference from far field sources. In one embodiment, the array 1605 is sized to have a maximum dimension D1 or D2 across the surface of the area occupied by the coils that is approximately 100% to 300% of a predetermined maximum sensing distance that the markers are to be spaced from the plane of the coils. Thus, the size of the array 1605 is determined by identifying the distance that the marker is to be spaced apart from the array to accurately measure the marker signal, and then arrange the coils so that the maximum dimension of the array is approximately 100% to 300% of that distance. The maximum dimension of the array 1605, for example, can be approximately 200% of the sensing distance at which a marker is to be placed from the array 1605. In one specific embodiment, the marker 40 has a sensing distance of 20 cm and the maximum dimension of the array of coils 1602 is between 20 cm and 60 cm, and more specifically 40 cm.

A coil array with a maximum dimension as set forth above is particularly useful because it inherently provides a filter that mitigates interference from far field sources. As such, one aspect of several embodiments of the invention is to size the array based upon the signal from the marker so that the array preferentially measures near field sources (i.e., the field generated by the marker) and filters interference from far field sources.

The coils 1602 are electromagnetic field sensors that receive magnetic flux produced by the wireless markers 40 and in turn produce a current signal representing or proportional to an amount or magnitude of a component of the magnetic field through an inner portion or area of each coil. The field component is also perpendicular to the plane of each coil 1602. Each coil represents a separate channel, and thus each coil outputs signals to one of 32 output ports 1606. A preamplifier, described below, may be provided at each output port 1606. Placing preamplifiers (or impedance buffers) close to the coils minimizes capacitive loading on the coils, as described herein. Although not shown, the sensing unit 1601 also includes conductive traces or conductive paths routing signals from each coil 1602 to its corresponding output port 1606 to thereby define a separate channel. The ports in turn are coupled to a connector 1608 formed on the panel 1604 to which an appropriately configured plug and associated cable may be attached.

The sensing unit 1601 may also include an onboard memory or other circuitry, such as shown by electrically erasable programmable read-only memory (EEPROM) 1610. The EEPROM 1610 may store manufacturing information such as a serial number, revision number, date of manufacture, and the like. The EEPROM 1610 may also store per-channel calibration data, as well as a record of run-time. The run-time will give an indication of the total radiation dose to which the array has been exposed, which can alert the system when a replacement sensing subsystem is required.

Although shown in one plane only, additional coils or electromagnetic field sensors may be arranged perpendicular to the panel 1604 to help determine a three-dimensional location of the wireless markers 40. Adding coils or sensors in other dimensions could increase the total energy received from the wireless markers 40, but the complexity of such an array would increase disproportionately. The inventors have found that three-dimensional coordinates of the wireless markers 40 may be found using the planar array shown in FIG. 26A-B.

Implementing the sensor assembly 1012 may involve several considerations. First, the coils 1602 may not be presented with an ideal open circuit. Instead, they may well be loaded by parasitic capacitance due largely to traces or conductive paths connecting the coils 1602 to the preamplifiers, as well as a damping network (described below) and an input impedance of the preamplifiers (although a low input impedance is preferred). These combined loads result in current flow when the coils 1602 link with a changing magnetic flux. Any one coil 1602, then, links magnetic flux not only from the wireless marker 40, but also from all the other coils as well. These current flows should be accounted for in downstream signal processing.

A second consideration is the capacitive loading on the coils 1602. In general, it is desirable to minimize the capacitive loading on the coils 1602. Capacitive loading forms a resonant circuit with the coils themselves, which leads to excessive voltage overshoot when the excitation source 1010 is energized. Such a voltage overshoot should be limited or attenuated with a damping or “snubbing” network across the coils 1602. A greater capacitive loading requires a lower impedance damping network, which can result in substantial power dissipation and heating in the damping network.

Another consideration is to employ preamplifiers that are low noise. The preamplification can also be radiation tolerant because one application for the sensor assembly 1012 is with radiation therapy systems that use linear accelerators (LINAC). As a result, PNP bipolar transistors and discrete elements may be preferred. Further, a DC coupled circuit may be preferred if good settling times cannot be achieved with an AC circuit or output, particularly if analog to digital converters are unable to handle wide swings in an AC output signal.

FIG. 27, for example, illustrates an embodiment of a snubbing network 1702 having a differential amplifier 1704. The snubbing network 1702 includes two pairs of series coupled resistors and a capacitor bridging therebetween. A biasing circuit 1706 allows for adjustment of the differential amplifier, while a calibration input 1708 allows both input legs of the differential amplifier to be balanced. The coil 1602 is coupled to an input of the differential amplifier 1704, followed by a pair of high-voltage protection diodes 1710. DC offset may be adjusted by a pair of resistors coupled to bases of the input transistors for the differential amplifier 1704 (shown as having a zero value). Additional protection circuitry is provided, such as ESD protection diodes 1712 at the output, as well as filtering capacitors (shown as having a 10 nF value).

c. Signal Processors and Controllers

The signal processor 1014 and the controller 1016 illustrated in FIG. 20 receive the signals from the sensor assembly 1012 and calculate the absolute positions of the markers 40 within the reference frame. Suitable signal processing systems and algorithms are set forth in U.S. application Ser. Nos. 10/679,801; 10/749,478; 10/750,456; 10/750,164; 10/750,165; 10/749,860; and 10/750,453, all of which are incorporated herein by reference.

### CONCLUSION

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to target locating and tracking systems, not necessarily the exemplary system generally described above.

The various embodiments described above can be combined to provide further embodiments. All the U.S. patents, U.S. patent application publications, U.S. patent applications, foreign patents, foreign patent applications, and non-patent publications referred to in this specification and/or listed in the Application Data Sheet are incorporated herein by reference, in their entirety. Aspects of the invention can be modified, if necessary, to employ systems, devices, and concepts of the various patents, applications, and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all target locating and monitoring systems that operate in accordance with the claims to provide apparatus and methods for locating, monitoring, and/or tracking the position of a selected target within a body. Accordingly, the invention is not limited, except as by the appended claims.

I/We claim:

1. An apparatus for facilitating radiation treatment of a target in a patient, comprising:
a conformal member contained in a cavity of a patient, the conformal member configured to be inserted into and releasably retained in a fixed relative position in the cavity of the patient; and
a marker associated with the conformal member, wherein the marker is retained in a fixed position in or on the conformal member.

2. The apparatus of claim 1 wherein the marker comprises a wireless transponder configured to wirelessly transmit a location signal in response to a wirelessly transmitted excitation energy.

3. The apparatus of claim 1 wherein the marker comprises a casing affixed in or on the conformal member and a magnetic transponder in the casing, and wherein the magnetic transponder comprises a coil and a capacitor coupled to the coil.

4. The apparatus of claim 1 wherein the conformal member comprises a mouthpiece, and wherein the apparatus further comprises a plurality of markers attached to the mouthpiece.

5. The apparatus of claim 4 wherein the markers comprise wireless transponders configured to wirelessly transmit location signals in response to wirelessly transmitted excitation energy.

6. The apparatus of claim 4 wherein the markers comprise a first magnetic transponder having a first resonant frequency and a second magnetic transponder having a second resonant frequency different than the first resonant frequency.

7. The apparatus of claim 4 wherein the markers comprise radiopaque elements.

8. The apparatus of claim 4 wherein the markers comprise magnetic transponders and/or radiographic fiducials.

9. The apparatus of claim 8 wherein the transponders and the fiducials are in a fixed relationship and/or orientation to one another.

10. The apparatus of claim 1 wherein the conformal member comprises a mouthpiece and the mouthpiece further comprises a first wall, a second wall and a base plate, wherein the first wall and the second wall extend upwardly from the base plate to form a channel and wherein at least a portion of the marker is in the base plate.

11. The apparatus of claim 1 wherein the conformal member is partially or fully constructed from a thermoplastic material.

12. The apparatus of claim 2 wherein the transponder comprises an alternating magnetic circuit having a ferrite core and a coil with a plurality of windings around the ferrite core.

13. The apparatus of claim 2 wherein the transponder comprises a ferrite core and a coil around the ferrite core, and wherein the marker further comprises a capsule encasing the transponder, the capsule having a longitudinal axis and a cross-sectional dimension normal to the longitudinal axis of not greater than 2 mm.

14. The apparatus of claim 1 wherein the conformal member further comprises a first marker in a first portion of the member and a second marker in a second portion of the member spaced apart from the first marker, wherein the first and the second markers are orthogonally oriented with respect to each other.

15. The apparatus of claim 1 wherein the marker comprises an alternating magnetic circuit and wherein the marker has a radiographic centroid and the alternating magnetic circuit has a magnetic centroid at least approximately coincident with the radiographic centroid.

16. A device for insertion into an oral cavity of a human, comprising:
a body configured to releasably affix to the teeth of a human, wherein the body is contained in the oral cavity of a human; and
a magnetic transponder including a circuit configured to be energized by a wirelessly transmitted pulsed magnetic field and to wirelessly transmit a pulsed magnetic location signal in response to the pulsed magnetic field, wherein the transponder is attached to the body.

17. The device of claim 16 further comprising a radiographic fiducial.

18. The device of claim 16 wherein the transponder and the fiducial are in a known orientation and location relative to each other.

19. The device of claim 16 wherein the transponder comprises an alternating magnetic circuit having a ferrite core and a coil with a plurality of windings around the ferrite core.

20. The device of claim 16 wherein the fiducial is made from gold, tungsten, platinum or other high-density metals.

21. The device of claim 16 wherein the transponder is encapsulated in the body and the transponder comprises an alternating magnetic circuit within the body, and wherein the transponder is not electrically coupled to external leads outside the body.

22. The device of claim 16 further comprising a second and a third transponder, wherein the first, second and third transponders are in a known position and orientation relative to each other and wherein at least one of the transponders is oriented orthogonal to the other two transponders.

23. A system for localizing and/or tracking a device contained in an oral cavity of a patient, comprising:
a body configured to be received in an oral cavity of a patient, the body having a channel configured to be retained by the patient's teeth and a magnetic marker having a transponder in or on the body, wherein the transponder has a circuit configured to be energized by a wirelessly transmitted pulsed magnetic field and to wirelessly transmit a pulsed magnetic location signal in response to the pulsed magnetic field;
an alignment device for aligning a head and/or neck of a patient during localizing and/or tracking of the transponder; and
an excitation source comprising an energy storage device, a source coil, and a switching network coupled to the energy storage device and the source coil, the source coil being configured to wirelessly transmit the pulsed magnetic field to energize the transponder, and the switching network being configured to alternately transfer (a) stored energy from the energy storage device to the source coil and (b) energy in the source coil back to the energy storage device.

24. The system of claim 23 wherein the switching network comprises an H-bridge switch.

25. The system of claim 23 wherein the switching network is configured to have a first on position in which the stored energy is transferred from the energy storage device to the source coil and a second on position in which energy in the source coil is transferred back to the energy storage device.

26. The system of claim 25 wherein the first on position has a first polarity and the second on position has a second polarity opposite the first polarity.

27. The system of claim 23 wherein the source coil comprises an array having a plurality of coplanar source coils.

28. The system of claim 27 wherein the switching network is configured to selectively energize the coplanar source coils to change a spatial configuration of the pulsed magnetic field.

29. The system of claim 23 wherein the transponder comprises an alternating magnetic circuit having a ferrite core and a coil with a plurality of windings around the ferrite core.

30. The system of claim 23 wherein the transponder is contained in the body, and wherein the transponder is not electrically coupled to external leads outside the body.

31. The system of claim 23 further comprising a radiographic fiducial in or on the body, wherein the transponder and the fiducial are in a fixed relative position and orientation relative to each other.

32. The system of claim 31 wherein the body further comprises a first portion and a second detachable portion, the first portion having a first and a second surface, the first surface having a channel configured to receive a patient's teeth, the second surface configured to mateably receive the second detachable portion, wherein the fiducials are positioned in or on the first portion and the transponders are positioned in or on a second portion.

33. A system for tracking a body contained in a cavity of a human, comprising:
a body configured to be received in a cavity of a human and a magnetic transponder contained in or on the body, wherein the transponder has a circuit configured to be energized by a wirelessly transmitted pulsed magnetic field and to wirelessly transmit a pulsed magnetic location signal in response to the pulsed magnetic field; and
a sensor assembly comprising a support member and a plurality of field sensors carried by the support member configured to sense the pulsed magnetic location signal from the transponder.

34. The system of claim 33 wherein the field sensors are responsive only to field components of the pulsed magnetic location signal normal to individual field sensors.

35. The system of claim 33 wherein the field sensors are arranged in an array occupying an area having a maximum dimension of approximately 100% to 300% of a predetermined sensing distance between the marker and the sensing array.

36. The system of claim 33 wherein the transponder comprises an alternating magnetic circuit having a ferrite core and a coil with a plurality of windings around the ferrite core.

37. A method for localizing a mouthpiece to facilitate radiation treatment of a target in a patient, comprising:
positioning the mouthpiece in an oral cavity of the patient, the mouthpiece configured to be received in the oral cavity of the patient and a marker associated with the mouthpiece; and
localizing the marker in the patient with respect to a target in the patient to facilitate radiation treatment of the target.

38. The method of claim 37 wherein localizing the marker comprises (a) wirelessly delivering a pulsed magnetic field to energize the marker, (b) wirelessly transmitting a pulsed location signal from the marker to a location outside the patient, (c) sensing the pulsed location signal at a sensor located outside the patient, and (d) periodically calculating a three-dimensional location of the marker in a reference frame.

39. The method of claim 38 further comprising providing an output of the location of the marker in the reference frame at least every t_{f} second and within tᵢ second from sensing the pulsed location signal, wherein t_{f} and tᵢ are not greater than 1 second.

40. The method of claim 39 wherein t_{f} and tᵢ are from approximately 10 ms to approximately 500 ms.

41. The method of claim 39 wherein providing an output of the location of the marker further comprises referencing the three-dimensional location of the marker with an image of the marker relative to a target.

42. The method of claim 37 wherein localizing the marker comprises determining whether the marker has moved from a desired location.

43. The method of claim 37 wherein localizing the marker occurs while delivering ionizing radiation to the target.

44. A mouthpiece for insertion into an oral cavity of a patient, comprising:
a unshaped body having a channel wherein the u-shaped body is made of a thermoplastic material such that a patient's teeth impressions may be fixedly defined in the channel;
a plurality of excitable markers fixable in or on the body at a known geometry relative to each other and relative to the target; and
a plurality of fiducials in or on the body, wherein the fiducials are radiographic.

45. The mouthpiece of claim 44 wherein the excitable markers are positioned substantially orthogonal to an adjacent marker.

46. The mouthpiece of claim 44 wherein the u-shaped body further comprises a first portion and a second detachable portion, the first portion having a first and a second surface, the first surface having the channel containing the patient's teeth impressions, the second surface configured to mateably receive the second detachable portion, wherein the fiducials are positioned in or on the first portion and the transponders are positioned in or on a second portion.

47. The mouthpiece of claim 44 wherein the excitable markers are a transponder having a circuit configured to be energized by a wirelessly transmitted magnetic excitation energy and to wirelessly transmit a magnetic location signal in response to the excitation energy.

48. The mouthpiece of claim 47 wherein the transponder comprises an alternating magnetic circuit having a ferrite core and a coil with a plurality of windings around the ferrite core.

49. The mouthpiece of claim 44 wherein the fiducial is made from gold, tungsten, platinum or other high-density metals.

50. The mouthpiece of claim 47 wherein the transponder is encapsulated in the u-shaped body and the transponder comprises an alternating magnetic circuit, and wherein the transponder is not electrically coupled to external leads outside the u-shaped body.
